(19) United States
(12) Patent Application Publication
(10) Pub. No.: US 2012/0219543 A1
(43) Pub. Date: Aug. 30, 2012

(54) METHODS AND PHARMACEUTICAL COMPOSITIONS FOR THE TREATMENT OF DISORDERS OF GLUCOSE HOMEOSTASIS

(76) Inventors: Raphael Scharfmann, Paris (FR); Michael Polak, Paris (FR); Samia Zertal, Paris (FR)

(21) Appl. No.: 13/502,784

(22) PCT Filed: Oct. 19, 2010
(86) PCT No.: PCT/EP2010/065675

§ 371 Date: May 11, 2012

(30) Foreign Application Priority Data
Oct. 20, 2009 (EP) 09305994.7

(51) Int. Cl. A61K 39/395; A61K 31/15; C12N 5/02; A61K 31/7088; C12Q 1/68; C12Q 1/02

(52) U.S. Cl. 424/130.1; 435/6.1; 435/29; 435/377; 514/44 R; 514/44 A; 514/664

(57) ABSTRACT

The invention is in the field of disorders of glucose homeostasis therapy. In particular the invention relates to a CFTR inhibitor or an inhibitor of CFTR gene expression for use in the treatment of disorders of glucose homeostasis. The present invention also relates to an in vitro methods for increasing the pool of Ngn3+ endocrine progenitor cells, pancreatic endocrine cells, or β cell mass obtained from stem cells, wherein said methods comprises the step of contacting stem cells with a CFTR inhibitor or an inhibitor of CFTR gene expression. The present invention also relates to a method of testing a subject thought to have or be predisposed to having disorders of glucose homeostasis, which comprises the step of analyzing a sample of interest from said subject for: (i) detecting the presence of a mutation in the CFTR gene and/or its associated promoter, and/or (ii) analyzing the expression of the CFTR gene.

### FIELD OF THE INVENTION

The invention is in the field of disorders of glucose homeostasis therapy. In particular the invention relates to a CFTR inhibitor for use in the treatment of disorders of glucose homeostasis.

### BACKGROUND OF THE INVENTION

Nowadays, more than 150 millions people suffer for diabetes in the world. This disease is rising heavily and it is estimated than in the next 20 years, 300 millions people could be affected.

Insulinotherapy is the large-scale diabetes mellitus treatment. It consists in recurrent injections of insulin everyday day. The hope is to replace this heavy treatment, which is associated with secondary effects, by a definitive cure. In order to cure diabetes, islets transplantation was tested. However, 5 to 10 organ donors are required to transplant a single diabetic patient. Thus, one of the major problem limiting islet transplantation therapy is the lack of organ donors.

An alternative source of beta cells is therefore required. Different approaches are being considered such as xenografts, transdifferentiation of bone narrow, liver or intestine cells, as well as differentiation of embryonic or adult stem cells, but they are still irrelevant for the treatment of diabetes mellitus.

Several studies have highlighted the importance of ATP-sensitive K+ channel (KATP channel) in the etiology of diabetes. Indeed, activating mutations in KCNJ11 gene, encoding for the Kir6.2 KATP channel subunit, are the most common cause of permanent neonatal diabetes mellitus (PNMD), accounting for the 38 to 50% of cases. The pancreatic beta cell KATP channel is composed of four inward rectifying K+ channel (Kir6.2) subunits, encoded by KCNJ11, and four sulfonylurea receptor (SUR1) encoded by ABCC8. This channel plays a key role in glucose stimulated-insulin secretion by regulating the flux of potassium ions across cell membranes. When blood glucose levels rise, the resulting increase in glucose metabolism results in a change in the ratio of cytosolic nucleotides [ADP]/[ATP], which causes closure of the KATP channel, leading to membrane depolarization. This subsequently activates voltage-dependent calcium channels and thus an influx of calcium triggering for insulin granule exocytose.

In animal models, the involvement of KATP channel subunits on endocrine pancreas development and function was dissected by gene targeting approach. Although SUR1 null mice were shown to be euglycemic with an islet histology nearly normal, they are markedly glucose-intolerant. These mice exhibit a transient NDM. Kir6.2 knockout newborn mice showed neither altered islet morphology nor severe defects in glucose-induced insulin secretion. However, as they grew, these KO mice exhibit impairment of glucose-dependant insulin secretion, a consequence of a dramatic reduction of beta cell mass due to an increase of beta cell apoptosis. These KO mice have also a marked increase of alpha cells suggesting strongly that KATP channels play an important role in insulin cell survival and endocrine pancreas differentiation. Although recent studies have highlighted the involvement of ionic channels activity in myoblast differentiation, little is know about the role of the KATP channels in the early steps of pancreas development. Furthermore, KATP channels activity (closure and opening) is known to be inhibited by pharmacological agents. Among them, the antidiabetic sulfonylureas that have been used since 1950's to restore the defective insulin secretion in patients with type 2 diabetes. The sulphonylureas bind with high affinity to the SUR1 receptor, specifically close the KATP channel in an ATP-independent manner and trigger insulin release. This useful strategy in type 2 diabetes was also tested in the treatment of diabetic patients with activating KCNJ11 mutations. Thus, in young patients, this sulphonylurea treatment (essentially with glibenclamide) replaced successfully insulin injections and allowed a prolonged and effective glycemic control. Treating PNMD with glibenclamide, not only ameliorates the glycemic control in comparison to insulin, but also improved the psychomotor development and the cognitive function of PNMD children with KCNJ11 mutations associated to severe neurologic forms.

Several clinical studies have established the efficiency of sulfonylurea replacement in PNMD children, demonstrating that pharmacogenomic approach is relevant to the improvement of diabetes therapy and quality of life of these patients diagnosed as early as 3 months (Sagen et al. 2004; Flechtner et al. 2006; Pearson et al. 2006; Codner et al. 2007; Begum-Hasan et al. 2008; Stoy et al. 2008). In most of cases, these young patients received glibenclamide at a median equivalent dose of 0.45 mg per kilogram per day (range, 0.05 to 1.5 mg per kilogram per day); this therapy being safe in the short term. Although data are available concerning the effects of glibenclamide therapy in type2 diabetic adult, less is known about its effects in the young PNMD patient, in the pancreas and specifically about the impact of glibenclamide treatment on beta cell specification and differentiation.

### SUMMARY OF THE INVENTION

The present invention relates to a CFTR inhibitor for use in the treatment of disorders of glucose homeostasis.

The present invention also relates to an inhibitor of CFTR gene expression for use in the treatment of disorders of glucose homeostasis.

The present invention also relates to a method for screening a drug for the treatment of disorders of glucose homeostasis, said method comprising contacting a test compound with a CFTR protein or gene and determining the ability of said test compound to inhibit the expression and/or activity of said gene or protein.

The present invention also relates to an in vitro method for increasing the pool of Ngn3+ endocrine progenitor cells obtained from stem cells, wherein said method comprises the step of contacting stem cells having the capacity to differentiate into pancreatic endocrine cells with a CFTR inhibitor or an inhibitor of CFTR gene expression.

The present invention also relates to an in vitro method for increasing the number of pancreatic endocrine cells obtained from stem cells, wherein said method comprises the step of contacting stem cells having the capacity to differentiate into pancreatic endocrine cells with a CFTR inhibitor or an inhibitor of CFTR gene expression.

The present invention also relates to an in vitro method for increasing the β cell mass obtained from stem cells, wherein said method comprises the step of contacting stem cells having the capacity to differentiate into pancreatic endocrine cells with a CFTR inhibitor or an inhibitor of CFTR gene expression.

The present invention also relates to an in vitro method for obtaining pancreatic endocrine cells, wherein said method comprises the step of contacting stem cells having the capacity to differentiate into pancreatic endocrine cells with a CFTR inhibitor or an inhibitor of CFTR gene expression.

The present invention also relates to a method of testing a subject thought to have or be predisposed to having disorders of glucose homeostasis, which comprises the step of analyzing a sample of interest from said subject for:

(i) detecting the presence of a mutation in the CFTR gene and/or its associated promoter, and/or

(ii) analyzing the expression of the CFTR gene.

### DETAILED DESCRIPTION OF THE INVENTION

The inventors' objective was to determine whether glibenclamide has deleterious effects on beta cell development. For this purpose, they use an in vitro model, which allows endocrine and acinar development from an embryonic Rat pancreases in a way that mimics pancreas development occurring in vivo (Attali et al. 2007). They studied the effects of increased concentrations of glibenclamide on pancreas development. They found that up to 1 micromolar, glibenclamide did not affect pancreas development. Interestingly, at higher concentrations, while pancreas morphology and cell proliferation rate was not modified, glibenclamide amplified the pool of endocrine progenitor expressing the transcription factor NGN3. This amplification was followed by the activation of the islet specific NeuroD1 transcription factor and a dramatic increase in beta cell mass. When used at high concentration (micromolar range), glibenclamide is known to inhibit CFTR (cystic fibrosis transmembrane regulator) channel (Schultz et al. 1996; Yamazaki and Hume 1997). They thus postulated that the observed effect of high concentration of Glibenclamide could be due to an off-target effect of glibenclamide on CFTR. To test this hypothesis, they cultured pancreases with Glycine hydrazide (GlyH101), a specific inhibitor of CFTR (Muanprasat et al. 2004). Importantly, they found that GlyH101 mimics glibenclamide effects. It increases the number of NGN3+ endocrine progenitor cells and the final number of beta cells that develop.

Taken together, they propose that CFTR inhibitors can be used to increase beta cell development.

### Methods of Treatment

Accordingly a first object of the present invention relates to a CFTR inhibitor for use in the treatment of disorders of glucose homeostasis.

As used herein, the term “disorders of glucose homeostasis” refers to conditions characterized by chronic excessive amount of glucose circulating in the blood plasma. This is generally a blood glucose level of 10+ mmol/L (180 mg/dl). The term is intended to encompass diabetes mellitus, Impaired Glucose Tolerance, gestational diabetes mellitus and cystic fibrosis-related diabetes. As used herein, the term “diabetes mellitus” is intended to encompass type 1 and type 2 diabetes mellitus.

In its broadest meaning, the term “treating” or “treatment” refers to reversing, alleviating, inhibiting the progress of, or preventing the disorder or condition to which such term applies, or one or more symptoms of such disorder or condition.

As used herein, the term “CFTR” has its general meaning in the art and refers to the cystic fibrosis transmembrane regulator protein. CFTR is a cAMP activated chloride channel expressed in epithelial cells in mammalian airways, intestine, pancreas and testis. CFTR is the chloride-channel responsible for cAMP-mediated chloride secretion. The term may include naturally occurring CFTRs and variants and modified forms thereof. The CFTR can be from zany source, but typically is a mammalian (e.g., human and non-human primate) CFTR, particularly a human CFTR.

A “CFTR inhibitor” as used herein has its general meaning in the art and is a compound that reduces the efficiency of ion transport by CFTR, particularly with respect to transport of chloride ions by CFTR. Preferably CFTR inhibitors of the invention are specific (i.e. selective) CFTR inhibitors, i.e., compounds that inhibit CFTR activity without significantly or adversely affecting activity of other ion transporters e.g., other chloride transporters, potassium transporters, and the like. Preferably the CFTR inhibitors are high-affinity CFTR inhibitors, e.g., have an affinity for CFTR of at least about one micromolar, usually about one to five micromolar. According to the invention, the CFTR inhibitor is not a sulfonylurea compound, and more particularly is not glibenclamide.

Typically, inhibition properties of a compound on CFTR activity may be evaluated by any method well known in the art. For example, compounds may be screened in a cell based assay of iodide influx after CFTR activation by an agonist mixture containing forskolin, IBMX and apigenin. Rates of iodide influx are then computed from the kinetics of fluorescence decrease following chloride replacement by iodide. The compounds selected as CFTR inhibitor are those that reduce iodide influx.

In one embodiment, the CFTR inhibitor (e.g. agonist, partial agonist or antagonist) is a low molecular weight antagonist, e.g. a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In one embodiment, CFTR inhibitors according to the invention are thiazolidinone compounds as described in Ma et al, 2002, J. Clin. Invest, 110:1651-1658.

In one embodiment, CFTR inhibitors according to the invention are hydrazide-containing compounds as described in the International Patent Application Publication WO 2005/094374 and Muanprasat et al. 2004 that are herein incorporated by reference. Typically the hydrazide-containing compounds comprise an aromatic- or heteroaromatic-substituted nitrogen, a hydrazide (which can be a glycine or oxamic hydrazide), and a substituted or substituted aryl group. In specific embodiments, the subject compounds are generally described by Formula (I) as follows:

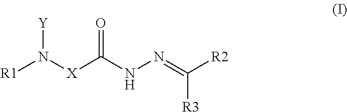

wherein:
X is independently chosen from an alkyl group, or a carbonyl group;
Y is independently chosen from an alky group; an alkyl group having polar substitutions, such as a sulfo group, or a carboxyl group, or a linker, such as an amide bond or an ether linker to provide for attachment of one or more larger polar molecules, such as a polyoxyalkyl polyether (such as a polyethylene glycol (PEG), polypropylene glycol, polyhydroxyethyl glycerol), disaccharides, a substituted or unsubstituted phenyl group, polyalkylimines, a dendrimer from 0-10 generation and the like, where Y can further include such an attached polar molecule(s);
R1 is independently chosen from a substituted or unsubstituted phenyl group, a substituted or unsubstituted heteroaromatic group such as a substituted or unsubstituted quinolinyl group, an substituted or unsubstituted anthracenyl group, and a substituted or unsubstituted naphthalenyl group;
R2 is a substituted or unsubstituted phenyl group; and
R3 is independently chosen from hydrogen and an alkyl group.

More particularly, the hydrazide-containing compounds may be selected from the group consisting of: N-2-napthalenyl-[(3:5-dibromo-2,4-dihydroxyphenyl)methylene]glycmehydrazide; N-2-napthalenyl-[(3,5-dibromo-2,4,6-trihydroxyphenyl)methylene]glycine hydrazide, N-(substituted-2-(napthalenyl)-[(3,5-dibromo-2,4-dihydroxyphenyl)methylene]glycine hydrazide, N-2-napthalenyl-[(3,5-dibromo-4-hydroxyphenyl)methylene]glycinehLydrazide; N-2-napthalenyl-[(3,5-dibromo-2-hydroxy-4-mthoxyphenyl)methylene]glycinehydrazide, N-1-napthalenyl-[(3,5-dibromo-2,4-dihydroxyphenyl)methylene]glycine hydrazide; N-1-napthalenyl-[(3,5-dibromo-2,4,6-trihydroxyphenyl)methylene]glycinehydrazide; N-(substituted-1-napthalenyl)-[(3,5-dibromo-2,4-dihydroxyphenyl)methylene]glycine hydrazide; N-1-napthalenyl-[(3,5-dibromo-4-hydroxyphenyl)methylene]glycine hydrazide; N-2-naptlialenyl-[(3,5-dibromo-2,4-dihydroxyphenyl)methylene]propionic acid hydrazide; N-2-napthalenyl-[(3,5-dibromo-4-hydroxyphenyl)methylene]propionic acid hydrazide; N-2-napthalenyl-[(3,5-dibromo-2,4-dihydroxyphenyl)ethylene]glycine hydrazide; N-2-napthalenyl-[(3,5-dibromo-4-hydroxyphenyl)ethylene]glycine hydrazide; N-2-napthalenyl-[(3,5-dibromo-2,4-dihydroxyphenyl)methylene]oxamic acid hydrazide; N-2-napthalenyl-[(3,5-dibromo-4-hydroxyphenyl)methylene]oxamic acid hydrazide; N-2-napthalenyl-[(3,5-dibromo-2,4-dihydroxyphenyl)ethylene]oxamic acid hydrazide; N-2-napthalenyl-[(3,5-dibromo-4-hydroxyphenyl)ethylene]oxamic acid hydrazide; 4-chlorophenyl-[(3,5-dibromo-4-hydroxyphenyl)methylene]glycine hydrazide; 4-chlorophenyl-[(3,5-dibromo-2,4-dihydroxyphenyl)methylene]glycine hydrazide; 4-methylphenyl-[(3,5-dibromo-2,4-dihydroxyphenyl)methylene]glycine hydrazide; 2-methylphenyl-[(3,5-dibromo-2,4-dihydroxyphenyl)methylene]glycine hydrazide; N-1-napthalenyl-[(3-bromo-4-hydroxyphenyl)methylene]glycine hydrazide; N-2-napthalenyl-[(2,4-dihydroxyphenyl)methylene]glycine hydrazide; N-2-napthalenyl-[(4-bromophenyl)methylene]glycine hydrazide; N′-2-napthalenyl-[(4-carboxyphenyl)methylene]glycine hydrazide; 4-chlorophenyl-[(3,5-dibromo-2-hdroxy-4-methoxyphenyl)methylene]glycine hydrazide; 4-chlorophenyl-[(2,4-dihydroxyphenyl)methylene]glycine hydrazide; N-2-anthracenyl-[(3,5-dibromo-2,4-dihydroxyphenyl)methylene]glycine hydrazide; N-2-anthracenyl-[(3,5-dibromo-4-hydroxyphenyl)methylene]glycine hydrazide; N-6-quinolmyl-[(3,5-dibromo-2,4-dihydroxyphenyl)methylene]glycine hydrazide; N-6-quinolinyl-[(3,5-dibromo-4-hydroxyphenyl)methylene]glycine hydrazide, N-(heteroaryl)-[(3,5-dibromo-4-hydroxyphenyl)methylene]glycine hydrazide; 2-naphthalenylamino-bis[(3,5-dibromo-2,4-dihydroxyphenyl)methylene]propanedioic acid dihydrazide; 2-naphthalenylamino-[(3,5-dibromo-2,4-dihydroxyphenyl)methylene][(2,4-disodiuni-disulfophenyl)methylene]propanedioic acid dihydrazide; 2-naphthalenylamino-[(3,5-dibromo-2,4-dihydroxyphenyl)methylene] [3-(4-sodium-sulfophenyl)-thioureido]propanedioic acid dihydrazide; 2-naphthalenylamino-[(3,5-dibromo-2,4-dihydroxyphenyl)methylene][3-[4-(3-(PEG)n-thioureido)phenyl)-thioureido]propanedioic acid dihydrazide; [2-(2-naphthalenylamino)-4-(PEG-amino)]butyric acid hydrazide; or 2-naphthalenylamino-[(3,5-dibromo-2,4-dihydroxyphenyl)methylene][3-[4-((3-(PEG)n-thioureido)phenyl-methyl)phenyl)-thioureido]propanedioic acid dihydrazide [MalH-(PEG)n B].

In a particular embodiment, the CFTR inhibitor according to the invention is N-2-napthalenyl-[(3,5-dibromo-2,4-dihydroxyphenyl)methylene]glycine hvdrazide also named as GlyH101.

In one embodiment, CFTR inhibitors according to the invention are those described in the International Patent Application Publication WO 2008/121877 that is herein incorporated by reference. In this specific embodiment, the subject compounds are generally described by Formula (II) as follows:

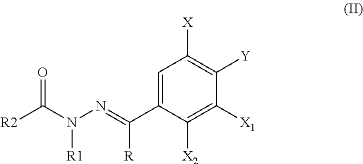

wherein:
R is selected from the group consisting of hydrogen, alkyl and substituted alkyl;
R1 is selected from the group consisting of hydrogen, alkyl, substituted alkyl, aryl, substituted aryl, alkenyl, substituted alkenyl, alkynyl, substituted alkynyl, cycloalkyl, substituted cycloalkyl, heterocyclic, substituted heterocyclic, heteroaryl and substituted heteroaryl;
or R and R1 together with the atoms bound thereto, form a heterocycle or substituted heterocycle;
R2 is selected from the group consisting of alkyl, substituted alkyl, aryl, substituted aryl, cycloalkyl, substituted cycloalkyl, heterocyclic, substituted heterocyclic, heteroaryl and substituted heteroaryl;
or R and R2 together with the atoms bound thereto, form a heterocycle or substituted heterocycle;
or R1 and R2 together with the atoms bound thereto, form a heterocycle or substituted heterocycle;
X and X1 are independently selected from the group consisting of hydrogen, halo, hydroxyl, nitro, alkyl, substituted alkyl, aryl, substituted aryl, alkoxy, substituted alkoxy, cycloalkyl, substituted cycloalkyl, heterocyclic, substituted heterocyclic, heteroaryl, substituted heteroaryl, carboxyl, and carboxyl ester;
X2 is selected from the group consisting of hydrogen, halo and hydroxyl; and
Y is selected from the group consisting of hydrogen, halo, hydroxyl, alkoxy and substituted alkoxy;
or either of X or X1 and Y together with the atoms bound thereto, form an aryl, substituted aryl, heteroaryl or substituted heteroaryl.

In a particular embodiment, the subject compounds may be selected from the group consisting of:
(E)-N′-(3,5-dibromo-4-hydroxybenzylidene)-2-hydroxy-2,2-diphenylacetohydrazide;
(E)-N′-(3,5-dibromo-4-hydroxybenzylidene)-2-(4-isobutylphenyl)propanehydrazide;
(E)-N′-(3,5-dibromo-4-hydroxybenzylidene)-3-(4-(trifluoromethoxy)phenoxy)benzohydrazide;
(E)-2-(4-chlorophenylamino)-N′-(3,5-diiodo-4-hydroxybenzylidene)acetohydrazide;
(E)-N′-(3,5-dibromo-4-hydroxybenzylidene)-3-(4-(trifluoromethyl)phenoxy)benzohydrazide;
(E)-N′-(3,5-dibromo-4-hydroxybeiizylidene)-2-(2,4-dichlorophenoxy)propanehydrazide;
(E)-3-(4-bromophenoxy)-N′-(3,5-dibromo-4-hydroxybenzylidene)benzohydrazide;
(E)-3-(3-methylphenoxy)-N′-(3,5-dibromo-4-hydroxybenzylidene)benzohydrazide;
(E)-4-bromo-3-chloro-N′-(3,5-dibromo-4-hydroxybeiizylidene)benzohydrazide;
(E)-N′-(3,5-dibromo-4-hydroxybeiizylidene)-2-(3,4-dichlorophenylamino)acetohydrazide;
(E)-2-(4-(1H-pyrrol-1-yl)phenoxy)-N′-(3,5-dibromo-4-hydroxybenzylidene)acetohydrazide;
(E)-2-(4-bromo-3,5-dimethylphenoxy)-N′-(3,5-dibromo-4-hydroxybenzylidene)acetohydrazide;
(E)-2-(6-bromonaphthalen-2-yloxy)-N′-(3,5-dibromo-4-hydroxybenzylidene)acetohydrazide;
(E)-N′-(3,5-dibromo-4-hydroxybenzylidene)-3-(4-fluoro-3-methoxyphenoxy)benzohydrazide;
(E)-N-allyl-N-(3,5-dibromo-4-hydroxybenzylidene)-3-phenoxybenzohydrazide;
(E)-N′-(1-(3,5-dibromo-4-hydroxyphenyl)ethylidene)-3-phenoxybenzohydrazide;
(E)-N′-(4-hydroxy-3,5-diiodobenzylidene)-3-phenoxybenzohydrazide;
(E)-N′-(3,5-dibromo-4-hydroxybenzylidene)-3-(3-(trifluoromethyl)phenoxy)benzohydrazide;
(E)-3-(benzyloxy)-N′-(3,5-dibromo-4-hydroxybenzylidene)benzohydrazide;
(E)-N′-(3,5-dibromo-4-hydroxybenzylidene)-3-phenoxybenzohydrazide;
(E)-N′-(3,5-dichloro-4-hydroxybenzylidene)-3-phenoxybenzohydrazide;
(E)-1-(3-chloro-5-(trifluoromethyl)pyridin-2-yl)-N′-(3,5-dibromo-4-hydroxybenzylidene)piperidine-4-carbohydrazide;
(E)-2-((4-chlorophenyl)(methyl)amino)-N′-(3,5-dibromo-4-hydroxybenzylidene)acetohydrazide;
(E)-N′-(3,5-dibromo-4-hydroxybeiizylidene)-2-p-tolylquinoline-4-carbohydrazide;
(E)-N′-(3,5-dibromo-4-hydroxybenzylidene)-3-(3,4-dichlorophenoxy)benzohydrazide;
(E)-2-(4-bromophenyl)-N′-(3,5-dibromo-4-hydroxybenzylidene)quino line-4-carbohydrazide;
(E)-N′-(3,5-dibromo-4-hydroxybenzylidene)-2′-fluorobiphenyl-4-carbohydrazide;
(E)-N′-(3,5-dibromo-4-hydroxybenzylidene)-2-(4-ethoxyphenyl)quino line-4-carbohydrazide;
(E)-2-(4-chloro-3-(trifluoromethyl)phenylamino)-N′-(3,5-dibromo-4-hydroxybenzylidene)acetohydrazide;
(E)-N′-(3,5-dibromo-4-hydroxybenzylidene)-2-(2,4-dichlorophenylamino)acetohydrazide;
(E)-3-(3-chloro-4-ethoxyphenoxy)-N′-(3,5-dibromo-4-hydroxybenzylidene)benzohydrazide;
(E)-3-bromo-4-chloro-N′-(3,5-dibromo-4-hydroxybenzylidene)benzohydrazide;
(E)-N′-(1-(3,5-dibromo-4-hydroxyphenyl)ethylidene)-2-(1OH-phenothiazin-10-yl)acetohydrazide;
(E)-N′-(3,5-dibromo-4-hydroxybenzylidene)-2-(naphthalen-1-yl)acetohydrazide;
(E)-N′-(3-bromo-4-hydroxy-5-iodobenzylidene)-3-phenoxybenzohydrazide;
(E)-N′-(3,5-dibromo-4-hydroxybenzylidene)-3-(3,4,5-trifluorophenoxy)benzohydrazide;
(E)-3-(3,5-bis(trifluoromethyl)phenoxy)-N′-(3,5-dibromo-4-hydroxybenzylidene)benzohydrazide;
(E)-N′-(3-bromo-5-chloro-4-hydroxybenzylidene)-3-phenoxybenzohydrazide;
(E)-8-chloro-N′-(3,5-dibromo-4-hydroxybenzylidene)-2-phenylquinoline-4-carbohydrazide;
(E)-2-(4-chlorophenyl)-N′-(3,5-dichloro-4-hydroxybenzylidene)quino line-4-carbohydrazide;
(E)-2-(4-chlorophenyl)-N′-(3,5-dibromo-4-hydroxybenzylidene)quino line-4-carbohydrazide;
(E)-N′-(3,5-dibromo-4-hydroxybenzylidene)-2-(2,3-dichlorophenylamino)acetohydrazide;
(E)-N′-(3,5-dibromo-4-hydroxybenzylidene)-2-(4-methoxyphenyl)quino line-4-carbohydrazide;
(E)-N′-(3,5-dibromo-4-hydroxybenzylidene)-3-(4-methoxy-3-methylphenoxy)benzohydrazide;
(E)-3-benzyl-N′-(3,5-dibromo-4-hydroxybenzylidene)benzohydrazide;
(E)-3′-((2-(2-hydroxy-2,2-diphenylacetyl)hydrazono)methyl)biphenyl-3-carboxylic acid;
(E)-N′-(3,5-dichloro-4-hydroxybenzylidene)-2-(10H-phenothiazin-10-yl)acetohydrazide;
(E)-N′-(3,5-dibromo-4-hydroxybenzylidene)-2-(thiophen-2-yl)quino line-4-carbohydrazide;
(E)-N′-(3,5-dibromo-4-hydroxybenzylidene)-2-(10H-phenothiazin-10-yl)acetohydrazide;
(E)-N′-(1-(3,5-dichloro-4-hydroxyphenyl)ethylidene)-3-phenoxybenzohydrazide;
(E)-2-(4-chlorophenyl)-N′-(4-hydroxy-3,5-diiodobenzylidene)quino line-4-carbohydrazide;
(E)-3-(3-acetylphenoxy)-N′-(3,5-dibromo-4-hydroxybenzylidene)benzohydrazide;
(E)-N′-(3,5-dibromo-4-hydroxybenzylidene)-3-(3-(trifluoromethoxy)phenoxy)benzohydrazide;
(E)-N′-(3,5-dibromo-4-hydroxybenzylidene)-3-(2,3-dihydrobenzo[b][1,4]dioxin-6-yloxy)benzohydrazide;
(E)-N′-(4-hydroxy-3,5-diiodobenzylidene)-2-(10H-phenothiazin-10-yl)acetohydrazide;
(E)-N′-(1-(3,5-dichloro-4-hydroxyphenyl)ethylidene)-2-(1OH-phenothiazin-10-yl)acetohydrazide;
(E)-N′-(1-(3,5-dibromo-4-hydroxyphenyl)-2-phenylethylidene)-3-phenoxybenzohydrazide;
(E)-3-benzoyl-N′-(3,5-dibromo-4-hydroxybenzylidene)benzohydrazide;
(E)-N′-(3,5-dibromo-4-hydroxybenzylidene)-3-(4-methoxyphenoxy)benzohydrazide;
(E)-3-chloro-N′-(3,5-dibromo-4-hydroxy benzylidene)-6-methoxybenzo[b]thiophene-2-carbohydrazide;
(E)-N′-(3,5-dibromo-4-hydroxybenzylidene)-4-phenoxybenzohydrazide;
(E)-N-(4-tert-butylbenzyl)-N′-(3,5-dibromo-4-hydroxybenzylidene)-3-phenoxybenzohydrazide;
(E)-N′-(3,5-dibromo-4-hydroxybenzylidene)-2-(5-methyl-2-phenylthiazol-4-yl)acetohydrazide;
(E)-2-(2-chloro-5-methylphenoxy)-N′-(3,5-dibromo-4-hydroxybenzylidene)acetohydrazide;
(E)-N′-(3,5-dibromo-4-hydroxybenzylidene)octanehydrazide;
(E)-4-(4-chlorophenylsulfonyl)-N′-(3,5-dibromo-4-hydroxybenzylidene)-3-methylthiophene-2-carbohydrazide;
(E)-2-(4-chlorophenylthio)-N′-(3,5-dibromo-4-hydroxybenzylidene)propanehydrazide;
(E)-N′-(3,5-difluoro-4-hydroxybenzylidene)-3-phenoxybenzohydrazide;
(E)-3,5-dichloro-N′-(3,5-dibromo-4-hydroxybenzylidene)benzohydrazide;
(E)-N′-(3,5-dibromo-4-hydroxybenzylidene)-N-methyl-3-phenoxybenzohydrazide;
(E)-3-(3-(benzyloxy)phenoxy)-N′-(3,5-dibromo-4-hydroxybenzylidene)benzohydrazide;
(E)-N-(3-(3-(2-(3,5-dibromo-4-hydroxybenzylidene)hydrazinecarbonyl)phenoxy)phenyl)methanesulfonamide;
(E)-2-(4-chlorophenyl)-N′-(3,5-dibromo-4-hydroxybenzylidene)isonicotinohydrazide;
(E)-2-(4-(3-chloro-5-(trifluoromethyl)pyridin-2-yl)piperazin-1-yl)-N′-(3,5-dibromo-4-hydroxybenzylidene)acetohydrazide;
(E)-N′-(3,5-dibromo-4-hydroxybenzylidene)-3-hydroxy-2-naphthohydrazide;
(E)-N′-(3,5-difluoro-4-hydroxybenzylidene)-2-(10H-phenothiazin-10-yl)acetohydrazide;
(E)-2-(4-chlorophenylamino)-N′-(3,5-dichloro-4-hydroxybenzylidene)acetohydrazide;
(E)-N′-(3,5-dibromo-4-hydroxybenzylidene)-2-(4-iodophenylamino)acetohydrazide;
(E)-N′-(3,5-dibromo-4-hydroxybenzylidene)-2-(4-(trifluoromethyl)phenylamino)acetohydrazide;
(E)-N′-(3,5-dibromo-4-hydroxybenzylidene)-2-(4-(trifluoromethoxy)phenylamino)acetohydrazide;
(E)-2-(3-chlorophenyl)-N′-(3,5-dibromo-4-hydroxybenzylidene)acetohydrazide;
(E)-N-(3-(2-(3,5-dibromo-4-hydroxybenzylidene)hydrazinecarbonyl)phenyl)-3-(trifluoromethyl)benzamide;
(E)-2-(4-chlorophenylamino)-N′-(1-(3,5-dibromo-4-hydroxyphenyl)ethylidene)acetohydrazide;
(E)-4-(benzyloxy)-N′-(3,5-dibromo-4-hydroxybenzylidene)benzohydrazide;
(E)-2-(1-bromonaphthalen-2-yloxy)-N′-(3,5-dibromo-4-hydroxybenzylidene)acetohydrazide;
(E)-N′-(3-bromo-4-hydroxy-5-nitrobenzylidene)-2-(4-chlorophenylammo)acetohydrazide;
(E)-N′-(3,5-dibromo-4-hydroxybenzylidene)-2-(2,4-dichlorophenoxy)acetohydrazide;
(E)-2-(4-bromophenoxy)-N′-(3,5-dibromo-4-hydroxybenzylidene)acetohydrazide;
(E)-3-chloro-N′-(3,5-dibromo-4-hydroxybenzylidene)benzo[b]thiophene-2-carbohydrazide;
(E)-N′-(3,5-dibromo-4-hydroxybenzylidene)-3-(o-tolyloxy)benzohydrazide;
(E)-3-tert-butyl-N′-(3,5-dibromo-4-hydroxybenzylidene)-1-(2,4-dichlorobenzyl)-1H-pyrazole-5-carbohydrazide;
(E)-2-(4-chlorophenyl)-N′-(3,5-dibromo-2,4-dihydroxybenzylidene)quino line-4-carbohydrazide;
(E)-N′-(3,5-dibromo-4-hydroxybenzylidene)-3-iodobenzohydrazide;
(E)-N′-(3,5-dibromo-4-hydroxybenzylidene)-3-(naphthalen-2-yloxy)benzohydrazide;
(E)-3-(biphenyl-3-yloxy)-N′-(3,5-dibromo-4-hydroxybenzylidene)benzohydrazide;
(E)-2-(5-chlorothiophen-2-yl)-N′-(3,5-dibromo-4-hydroxybenzylidene)quino line-4-carbohydrazide;
(E)-N′-(3,5-dibromo-4-hydroxybenzylidene)-5-(hex-1-ynyl)nicotinohydrazide;
(E)-N′-(3,5-dibromo-4-hydroxybenzylidene)-3-(3-(methoxymethyl)phenoxy)benzohydrazide;
(E)-4-tert-butyl-N′-(3,5-dibromo-4-hydroxybenzylidene)benzohydrazide;
(E)-N′-(3,5-dibromo-4-hydroxybenzylidene)-3-(4-isopropoxyphenoxy)benzohydrazide;
(E)-3-(4-chlorophenylsulfonyl)-N′-(3,5-dibromo-4-hydroxybenzylidene)thiazolidine-2-carbohydrazide;
(E)-3-tert-butyl-N′-(3,5-dibromo-4-hydroxybenzylidene)-1-(4-fluorobenzyl)-1Hpyrazole-5-carbohydrazide;
(E)-N′-(3,5-dibromo-4-hydroxybenzylidene)-2-(2,2-dimethyl-2,3-dihydrobenzofuran-7-yloxy)acetohydrazide;
(E)-N′-(3,5-dibromo-4-hydroxybenzylidene)-2,5-bis(2,2,2-trifluoroethoxy)benzohydrazide;
(E)-N′-(3,5-dibromo-4-hydroxybenzylidene)-2-phenoxybenzohydrazide;
(E)-N′-(3,5-dibromo-4-hydroxybenzylidene)-2-(7-methyl-2,3-dihydro-1H-inden-4-yloxy)acetohydrazide;
(E)-4-(4-chlorophenyl)-N′-(3,5-dibromo-4-hydroxybenzylidene)cyclohexanecarbohydrazide;
(E)-3,4-dichloro-N-(3-(2-(3,5-dibromo-4-hydroxybenzylidene)hydrazinecarbonyl)phenyl)benzenesulfonamide;
(E)-N′-(3,5-dibromo-4-hydroxybenzylidene)-2-(2,3-dichlorophenoxy)acetohydrazide;
(E)-2-(4-chlorophenylamino)-N′-(4-hydroxy-3-(trifluoromethoxy)benzylidene)acetohydrazide;
(E)-4-bromo-2-chloro-N′-(3,5-dibromo-4-hydroxybenzylidene)benzohydrazide;
(E)-N′-(3,5-dibromo-4-hydroxybenzylidene)-4-(trifluoromethoxy)benzohydrazide;
(E)-2-(4-chlorophenylamino)-N′-(3,4-dihydroxybenzylidene)acetohydrazide;
(E)-N′-(3,5-dibromo-4-hydroxybenzylidene)-2-(naphthalen-1-yloxy)acetohydrazide;
(E)-N′-(3,4-dihydroxybenzylidene)-3-(4-fluorobenzyloxy)thiophene-2-carbohydrazide;
(E)-N′-(3,5-dibromo-4-hydroxybenzylidene)-2-naphthohydrazide;
(E)-N-(3-(2-(3,4-dihydroxybenzylidene)hydrazinecarbonyl)phenyl)naphthalene-2-sulfonamide;
(E)-N′-(3,5-dibromo-4-hydroxybenzylidene)-4-(trifluoromethyl)benzohydrazide;
(E)-3-bromo-N′-(3,5-dibromo-4-hydroxybenzylidene)benzohydrazide;
(E)-N′-(3,5-dibromo-4-hydroxybenzylidene)-3-(4-(pyrrolidine-lcarbonyl)phenoxy)benzohydrazide;
(E)-N′-(3,5-dibromo-4-hydroxybenzylidene)-1H-indole-2-carbohydrazide;
(E)-3-chloro-N′-(3,5-dibromo-4-hydroxybenzylidene)benzohydrazide;
(E)-N′-(3,5-dibromo-4-hydroxybenzylidene)-2-(4-ethylphenoxy)acetohydrazide;
(E)-3-chloro-N′-(3,5-dibromo-4-hydroxybenzylidene)-6-methylbenzo[b]thiophene-2-carbohydrazide;
(E)-ethyl-1-(2-(2,6-dibromo-4-((2-(3-phenoxybenzoyl)hydrazono)methyl)phenoxy)acetyl)piperidine-4-carboxylate;
(E)-N′-(3,5-dibromo-4-hydroxybenzylidene)-2-(4-(trifluoromethoxy)phenoxy)acetohydrazide;
(E)-N′-(3,5-dibromo-4-hydroxybenzylidene)-5-methyl-1-(4-methylphenyl)-1Hpyrazole-4-carbohydrazide;
(E)-N′-(3,5-dibromo-4-hydroxybenzylidene)-4-methyl-2-phenylpyrimidine-5-carbohydrazide;
(E)-N′-(3,5-dibromo-4-hydroxybenzylidene)-3-(trifluoromethyl)benzohydrazide;
(E)-2-(3-chlorophenylamino)-N′-(3,5-dibromo-4-hydroxybenzylidene)acetohydrazide;
(E)-N-(3-(2-(3,5-dibromo-4-hydroxybenzylidene) hydrazinecarbonyl)phenyl)benzenesulfonamide;
(E)-2-(4-chlorophenylamino)-N′-(4-hydroxy-3-(trifluoromethyl)benzylidene)acetohydrazide;
(E)-N′-(3,5-dibromo-4-hydroxybenzylidene)-4-(2,5-dimethyl-1H-pyrrol-lyl)benzohydrazide;
(E)-2-(2,6-dibromo-4-((2-(3-phenoxybenzoyl)hydrazono)methyl)phenoxy)acetic acid;
(E)-tert-butyl 1-(2-(3,5-dibromo-4-hydroxybenzylidene)hydrazinyl)-1-oxo-3-phenylpropan-2-ylcarbamate;
(E)-N′-(3,5-dibromo-4-hydroxybenzylidene)-1-phenyl-5-(trifluoromethyl)-1Hpyrazole-4-carbohydrazide;
(E)-1-(4-chlorophenyl)-N′-(3,5-dibromo-4-hydroxybenzylidene)-5-propyl-1Hpyrazole-4-carbohydrazide;
(E)-2-(7-chloroquinolin-4-ylthio)-N′-(3,5-dibromo-4-hydroxybenzylidene)acetohydrazide;
(E)-N′-(3,5-dibromo-4-hydroxybenzylidene)-3-(4-(hydroxymethyl)phenoxy)benzohydrazide;
(E)-N′-(3,5-dibromo-4-hydroxybenzylidene)-5-((4,5-dichloro-1H-imidazol-1-yl)methyl)furan-2-carbohydrazide;
(E)-4-chloro-N′-(3,5-dibromo-4-hydroxybenzylidene)benzohydrazide;
(E)-2-(6-bromonaphthalen-2-yloxy)-N′-(4-hydroxy-3,5-di(thiophen-3-yl)benzylidene)acetohydrazide;
(E)-3-chloro-N′-(3,5-dibromo-4-hydroxybenzylidene)-4-methylthiophene-2-carbohydrazide;
(E)-N′-(3,5-dibromo-4-hydroxybenzylidene)-3,5-bis(trifluoromethyl)benzohydrazide;
(E)-2-(4-chlorophenylamino)-N′-(3,4,5-trifluorobenzylidene)acetohydrazide;
(E)-N′-(3,5-dibromo-4-hydroxybenzylidene)-4-methyl-2-phenylthiazole-5-carbohydrazide;
(E)-N′-(3,5-dibromo-4-hydroxybenzylidene)-4-phenyl-1,2,3-thiadiazole-5-carbohydrazide;
(E)-N′-(3,5-dibromo-4-hydroxybenzylidene)-2-phenylthiazole-4-carbohydrazide;
(E)-2-(2-chlorophenoxy)-N′-(3,5-dibromo-4-hydroxybenzylidene)acetohydrazide;
(E)-N′-(3,5-dibromo-4-hydroxybenzylidene)-2-(2,3-dichlorophenyl)thiazole-4-carbohydrazide;
(E)-2-(4-chloro-2-methylphenoxy)-N′-(3,5-dibromo-4-hydroxybenzylidene)acetohydrazide;
(E)-N′-((1H-indol-6-yl)methylene)-2-(4-chlorophenylamino)acetohydrazide;
(E)-N′-(3,5-dibromo-4-hydroxybenzylidene)biphenyl-4-carbohydrazide;
(E)-N′-(3,5-dibromo-4-hydroxybenzylidene)-2-phenylacetohydrazide;
(E)-2-(4-(1,3-dithiolan-2-yl)phenoxy)-N′-(3,5-dibromo-4-hydroxybenzylidene)acetohydrazide;
(E)-N′-(3,5-dibromo-4-hydroxybenzylidene)benzo[b]thiophene-2-carbohydrazide;
(E)-2-(2-chlorophenylamino)-N′-(3,5-dibromo-4-hydroxybenzylidene)acetohydrazide;
(E)-N′-(3-bromo-4-fluorobenzylidene)-2-(4-chlorophenylamino)acetohydrazide;
(E)-N′-(3,5-dibromo-4-hydroxybenzylidene)-4H-thieno[3,2-c]chromene-2-carbohydrazide;
(E)-2-(4-chlorophenylamino)-N′-(4-fluoro-3-methylbenzylidene)acetohydrazide;
(E)-2-(4-chlorophenylamino)-N′-(4-fluorobenzylidene)acetohydrazide;
(E)-N′-(3,5-dibromo-4-hydroxybenzylidene)-5-methyl-4-phenyloxazole-2-carbohydrazide;
(E)-2-(4-chlorophenylsulfonyl)-N′-(3,5-dibromo-4-hydroxybenzylidene)acetohydrazide;
(E)-N′-(3,5-dibromo-4-hydroxybenzylidene)-2-(3-phenoxyphenylamino)acetohydrazide;
(3-bromo-4-chlorophenyl)(3-(3,5-dibromo-4-hydroxyphenyl)-4,5-dihydro-1Hpyrazol-1-yl)methanone;
6-(3,5-dibromo-4-hydroxyphenyl)-2-(3-hydroxybenzyl)-4,5-dihydropyridazin-3(2H)-one;
(E)-2-benzyl-3-(3,5-dibromo-4-hydroxybenzylideneamino)quinazolin-4(3H)-one;

and
(E)-3-(3,5-dibromo-4-hydroxybenzylideneamino)-2-isopropylquinazolin-4(3H)-10 one.

Alternatively, the CFTR inhibitor may consist in an antibody (the term including “antibody fragment”). In particular, the CFTR inhibitor may consist in an antibody directed against the CFTR, in such a way that said antibody inhibits the efficiency of ion transport by CFTR

Antibodies can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see, e.g., U.S. Pat. No. 4,946,778) can be adapted to produce anti-CFTR single chain antibodies. The CFTR inhibitor (e.g. agonist, partial agonist or antagonist) useful in practicing the present invention also include anti-CFTR antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to CFTR.

Humanized antibodies and antibody fragments thereof can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

Then after raising antibodies as above described, the skilled man in the art can easily select those modulating the CFTR.

In another embodiment the CFTR inhibitor is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S.D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then after raising aptamers directed against the CFTRs as above described, the skilled man in the art can easily select those modulating the CFTR.

A further object of the invention relates to an inhibitor of CFTR gene expression for use in the treatment of disorders of glucose homeostasis.

An “inhibitor of expression” refers to a natural or synthetic compound that has a biological effect to inhibit or significantly reduce the expression of a gene.

Inhibitors of expression for use in the present invention may be based on anti-sense oligonucleotide constructs. Anti-sense oligonucleotides, including anti-sense RNA molecules and anti-sense DNA molecules, would act to directly block the translation of CFTR mRNA by binding thereto and thus preventing protein translation or increasing mRNA degradation, thus decreasing the level of CFTR, and thus activity, in a cell. For example, antisense oligonucleotides of at least about 15 bases and complementary to unique regions of the mRNA transcript sequence encoding CFTR can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of expression for use in the present invention. CFTR gene expression can be reduced by contacting a subject or cell with a small double stranded RNA (dsRNA), or a vector or construct causing the production of a small double stranded RNA, such that CFTR gene expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836). All or part of the phosphodiester bonds of the siRNAs of the invention are advantageously protected. This protection is generally implemented via the chemical route using methods that are known by art. The phosphodiester bonds can be protected, for example, by a thiol or amine functional group or by a phenyl group. The 5′- and/or 3′-ends of the siRNAs of the invention are also advantageously protected, for example, using the technique described above for protecting the phosphodiester bonds. The siRNAs sequences advantageously comprises at least twelve contiguous dinucleotides or their derivatives.

As used herein, the term “siRNA derivatives” with respect to the present nucleic acid sequences refers to a nucleic acid having a percentage of identity of at least 90% with erythropoietin or fragment thereof, preferably of at least 95%, as an example of at least 98%, and more preferably of at least 98%.

As used herein, “percentage of identity” between two nucleic acid sequences, means the percentage of identical nucleic acid, between the two sequences to be compared, obtained with the best alignment of said sequences, this percentage being purely statistical and the differences between these two sequences being randomly spread over the nucleic acid acids sequences. As used herein, “best alignment” or “optimal alignment”, means the alignment for which the determined percentage of identity (see below) is the highest. Sequences comparison between two nucleic acids sequences are usually realized by comparing these sequences that have been previously align according to the best alignment; this comparison is realized on segments of comparison in order to identify and compared the local regions of similarity. The best sequences alignment to perform comparison can be realized, beside by a manual way, by using the global homology algorithm developed by SMITH and WATERMAN (Ad. App. Math., vol. 2, p:482, 1981), by using the local homology algorithm developed by NEDDLEMAN and WUNSCH (J. Mol. Biol., vol. 48, p:443, 1970), by using the method of similarities developed by PEARSON and LIPMAN (Proc. Natl. Acd. Sci. USA, vol. 85, p:2444, 1988), by using computer softwares using such algorithms (GAP, BESTFIT, BLAST P, BLAST N, FASTA, TFASTA in the Wisconsin Genetics software Package, Genetics Computer Group, 575 Science Dr., Madison, Wis. USA), by using the MUSCLE multiple alignment algorithms (Edgar, Robert C., Nucleic Acids Research, vol. 32, p:1792, 2004). To get the best local alignment, one can preferably used BLAST software. The identity percentage between two sequences of nucleic acids is determined by comparing these two sequences optimally aligned, the nucleic acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

shRNAs (short hairpin RNA) can also function as inhibitors of expression for use in the present invention.

Ribozymes can also function as inhibitors of expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of CFTR mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable.

Both antisense oligonucleotides and ribozymes useful as inhibitors of expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and preferably cells expressing CFTR. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adenoviruses and adeno-associated (AAV) viruses, which are double-stranded DNA viruses that have already been approved for human use in gene therapy. Actually 12 different AAV serotypes (AAV1 to 12) are known, each with different tissue tropisms (Wu, Z Mol Ther 2006; 14:316-27). Recombinant AAV are derived from the dependent parvovirus AAV2 (Choi, V W J Virol 2005; 79:6801-07). The adeno-associated virus type 1 to 12 can be engineered to be replication deficient and is capable of infecting a wide range of cell types and species (Wu, Z Mol Ther 2006; 14:316-27). It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, siRNA, shRNA or ribozyme nucleic acid sequence is under the control of a heterologous regulatory region, e.g., a heterologous promoter. The promoter may be specific for Muller glial cells, microglia cells, endothelial cells, pericyte cells and astrocytes For example, a specific expression in Muller glial cells may be obtained through the promoter of the glutamine synthetase gene is suitable. The promoter can also be, e.g., a viral promoter, such as CMV promoter or any synthetic promoters.

A further object of the invention relates to a method for the treatment of disorders of glucose homeostasis comprising administering a subject in need thereof with an active ingredient of the invention as above described (CFTR inhibitors or inhibitors of CFTR gene expression).

The term “subject” means a member of any mammalian or non-mammalian species that may have a need for the pharmaceutical methods, compositions and treatments described herein. Subjects thus include, without limitation, primate (including humans), canine, feline, ungulate (e.g., equine, bovine, swine (e.g., pig)), avian, and other subjects. Humans and non-human animals having commercial importance (e.g., livestock and domesticated animals) are of particular interest.

Active ingredients of the invention may be administered in the form of a pharmaceutical composition, as defined below.

Preferably, said active ingredient in a therapeutically effective amount.

By a “therapeutically effective amount” is meant a sufficient amount of the active ingredient to treat disorders of glucose homeostasis at a reasonable benefit/risk ratio applicable to any medical treatment.

It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Screening Methods

The present invention also provides novel targets and methods for the screening of drug candidates or leads. Such drug candidates or leads are useful for developing a treatment for disorders of glucose homeostasis. The methods include binding assays and/or functional assays, and may be performed in vitro, in cell systems, in animals, etc. Functional assays comprise, but are not limited to a cell based assay of iodide influx after CFTR activation by an agonist mixture containing forskolin, IBMX and apigenin. The in vitro assays, cell-based assays and animal-based assays involve a CFTR protein.

The invention relates to methods for screening of compounds that inhibit the CFTR activity.

Therefore, the present invention concerns a method for screening a drug for the treatment of disorders of glucose homeostasis, said method comprising contacting a test compound with a CFTR protein or gene and determining the ability of said test compound to inhibit the expression and/or activity of said gene or protein.

In a further particular embodiment, the method comprises contacting a recombinant host cell expressing CFTR with a test compound, and determining the ability of said test compound to bind CFTR and/or to inhibit the activity of CFTR.

The determination of binding may be performed by various techniques, such as by labelling of the test compound, by competition with a labelled reference ligand, two-hybrid Screening Assay, etc. Modulation of activity includes, without limitation, iodide efflux after CFTR activation by an agonist mixture containing forskolin, IBMX and apigenin.

The invention also concerns methods of selecting biologically active compounds using non-human transgenic animals expressing a CFTR protein. Said methods comprise (i) administrating a test compound to said non-human transgenic animal, and (ii) determining the ability of said test compound to inhibit the CFTR activity.

The above screening assays may be performed in any suitable device, such as plates, tubes, dishes, flasks, etc. Typically, the assay is performed in multi-wells plates. Several test compounds can be assayed in parallel.

Furthermore, the test compound may be of various origin, nature and composition. It may be any organic or inorganic substance, such as a lipid, peptide, polypeptide, nucleic acid, small molecule, etc., in isolated or in mixture with other substances. The compounds may be all or part of a combinatorial library of products, for instance. The test compounds can be an antisense or an RNAi. The test compounds can be competitive or suicide substrates. By “suicide substate” is intended a compounds that, after binding CFTR protein, the reactive group forms an irreversible bond with CFTR rendering it inactive.

### Methods of Differentiation

A further object of the invention relates to an in vitro method for increasing the pool of Ngn3+ endocrine progenitor cells obtained from stem cells, wherein said method comprises the step of contacting stem cells having the capacity to differentiate into pancreatic endocrine cells with a CFTR inhibitor or an inhibitor of CFTR gene expression.

In a further aspect, the present invention provides an in vitro method for increasing the number of pancreatic endocrine cells obtained from stem cells, wherein said method comprises the step of contacting stem cells having the capacity to differentiate into pancreatic endocrine cells with a CFTR inhibitor or an inhibitor of CFTR gene expression.

In another aspect, the present invention also relates to an in vitro method for increasing the β cell mass obtained from stem cells, wherein said method comprises the step of contacting stem cells having the capacity to differentiate into pancreatic endocrine cells with a CFTR inhibitor or an inhibitor of CFTR gene expression.

The present invention also relates to an in vitro method for obtaining pancreatic endocrine cells, wherein said method comprises the step of contacting stem cells having the capacity to differentiate into pancreatic endocrine cells with a CFTR inhibitor or an inhibitor of CFTR gene expression.

In a further aspect, the present invention provides the use of a CFTR inhibitor or an inhibitor of CFTR gene expression for the in vitro or ex vivo differentiation of stem cells into pancreatic endocrine cells.

As used herein, the term “Ngn3+ endocrine progenitor cells” refers to precursors of pancreatic endocrine cells expressing the transcription factor Neurogenin-3 (Ngn3). Progenitor cells are more differentiated than multipotent stem cells and can differentiate into only few cell types. In particular, Ngn3+ endocrine progenitor cells have the ability to differentiate into the five pancreatic endocrine cell types (α, β, δ, e and PP). The expression of Ngn3 may be assessed by any method known by the skilled person such as immunochemistry using an anti-Ngn3 antibody or quantitative RT-PCR.

The term “stem cells” refers to cells which have the ability to go through numerous cycles of cell division while maintaining an undifferentiated state and have the capacity to differentiate into specialized cell types. There are two broad types of mammalian stem cells: embryonic stem cells isolated from the blastocysts and adult stem cells found in adult tissues.

Stem cells may be classified according to their potency (their ability to differentiate into different cell types). Totipotent stem cells can differentiate into embryonic and extraembryonic cell types. Such cells contain all the genetic information needed to create a complete and viable organism. Pluripotent stem cells can differentiate into nearly all cell types but cannot develop into an embryo. These cells maintain the plasticity to generate all types of cells in an individual, except extraembryonic tissue such as placenta. Multipotent stem cells can differentiate into a number of cell types, but only those of a closely related family of cells. Adult stem cells, which reside in small number in almost all adult tissues, are generally multipotent: their regenerative potential is tissue or germ-layer specific.

As used herein, the term “stem cells” encompasses embryonic stem cells, adult stem cells and reprogrammed somatic cells (induced pluripotent stem cells also named IPS). In a particular embodiment, embryonic stem cells are non-human embryonic stem cells.

In an embodiment, stem cells having the capacity to differentiate into pancreatic endocrine cells are selected from the group consisting of pancreatic stem cells, pluripotent stem cells and multipotent stem cells.

In a particular embodiment, pancreatic stem cells are selected from the group consisting of stem cells derived from pancreatic islets, pancreatic ducts, pancreatic acinar cells and stem cells derived from the dorsal pancreatic bud from embryos.

As used herein, the term “cells derived from” shall be taken to indicate that this particular group of cells has originated from the specified source, but has not necessarily been obtained directly from said source.

As used herein, the term “pancreatic stem cells” refers to multipotent and organ specific stem cells expressing Pdx1 and which are able to differentiate into all types of pancreatic cells. The pancreas duodenal homeobox gene Pdx1 (UniGene Hs.32938) is one of the earliest genes expressed in the developing pancreas. Cells expressing Pdx1 give rise to all three types of pancreatic tissue, exocrine, endocrine and duct. After birth, Pdx1 expression is essentially restricted to β cells within the endocrine islets of the pancreas.

The identification of pancreatic stem cells from pancreatic islet and ductal populations has been described in Seaberg et al. (Seaberg et al., 2004). This paper demonstrated that these stem cells coexpress neural and pancreatic precursor markers and differentiate to form distinct populations of neurons, glial and stellate cells, pancreatic endocrine beta-, alpha- and delta-cells, and pancreatic exocrine cells.

Furthermore, it was recently found that pancreatic ductal and acinar cells are able under certain conditions to regress to a less differentiated phenotype and then can differentiate to form endocrine cells and, in particular, to form β cells (Bonner-Weir et al., 2008; Minami et al., 2008).

Consequently, stem cells having the capacity to differentiate into pancreatic endocrine cells may be pancreatic stem cells derived from exocrine, endocrine or ductal tissue or differentiated pancreatic cells which move into a less differentiated stage to express Pdx1.

Such pancreactic stem cells may be obtained from adult tissue by any method known in the prior art such as those described in the articles of Seaberg et al. Bonner-Weir et al., and Minami et al. (Seaberg et al., 2004 Bonner-Weir et al., 2008; Minami et al., 2008). Pancreatic stem cells may also be derived from the dorsal pancreatic bud from embryos. The dorsal pancreas is an embryonic bud from the endodermal lining of the gut on the dorsal wall cephalad to the level of the hepatic diverticulum, which forms most of the pancreas and its main duct. Pancreatic stem cells expressing Pdx1 may be obtained from fertilized ovocytes when pancreatic tissue has started to develop and before the terminal differentiation of most pancreatic cells.

In an embodiment, pancreatic stem cells are derived from human embryos. The age of these embryos is between 2 and 12 weeks of development, preferably between 2 and 8 weeks and more preferably between 2 and 6 weeks of development.

In a particular embodiment, pancreatic stem cells are derived from the dorsal pancreatic bud from non-human embryos due to some patent law and practices.

In another embodiment, stem cells having the capacity to differentiate into pancreatic endocrine cells are multipotent stem cells derived from other adult tissue than pancreatic tissue. Preferably, multipotent stem cells are derived from adult tissue selected from the group consisting of bone marrow, liver, central nervous system, spleen and adipose tissue.

Bone marrow-derived stem cells (hematopoietic or mesenchymal) have been described to be able to differentiate into pancreatic endocrine cells (Oh et al., 2004; Moriscot et al., 2005; Sun et al., 2007; Gabr et al., 2008). Bone marrow-derived stem cells may be isolated from the bone marrow based on their ability to adhere to plastic support. Then, they may be expanded and cultured. Pdx-1 gene expression may be induced in these cells using factors such as dimethyl sulfoxide, trichostatin or β-mercaptoethanol.

Mesenchymal stem cells from bone marrow and adipose tissue represent a very similar cell population with comparable phenotype. Consequently, adipose tissue-derived mesenchymal stem cells have also the potential to differentiate in pancreatic endocrine cells (Timper et al., 2006).

Liver stem cells, also named oval stem cells, have been described to be able to differentiate into pancreatic endocrine cells when cultured in a high-glucose environment (Yang et al., 2002). Another possibility may be to induce transdifferentiation of liver stem cells into pancreatic progenitor cells by expressing a Pdx-1 transgene (Sapir et al., 2005).

Brain-derived neural progenitor cells (Hori et al., 2005) and splenocytes (Kodama et al., 2003) have been also described to be able to differentiate into pancreatic endocrine cells.

In a preferred embodiment, multipotent stem cells derived from adult tissue and having the capacity to differentiate into pancreatic endocrine cells are not genetically modified. These cells exhibit the capacity to differentiate into pancreatic endocrine cells only by culturing them in presence of specific growth factors and/or compounds.

In a further particular embodiment, stem cells are pluripotent stem cells obtained by reprogramming of somatic cells. Such cells are also named induced pluripotent stem cells or IPS.

It has been found that induced pluripotent stem cells recapitulated the features of embryonic stem cells, such as human embryonic stem cells, and are thus an alternative to the controversial use of these cells (Romano et al., 2008). Induced pluripotent stem cells may be obtained from somatic cells, such as human skin fibroblasts, by a variety of methods essentially based on manipulation of a selected group of transcription factors (Maherali et al., 2008). For instance, induced pluripotent stem cells have been generated by ectopic expression of four transcription factors, OCT4, SOX2, KLF4 and c-MYC 10 (Takahashi et al., 2007; Lowry et al., 2008) or OCT4, SOX2, NANOG and LIN28 (Yu et al., 2007). Furthermore, it has been demonstrated that induced pluripotent cells have the potential to differentiate into pancreatic endocrine cells (Tateishi et al., 2008).

In another embodiment, pluripotent stem cells are derived from embryonic stem cells. Embryonic stem (ES) cells are derived from totipotent cells of the early mammalian embryo and are capable of unlimited, undifferentiated proliferation in vitro. Essential characteristics of these cells include (i) derivation from the preimplantation or periimplantation embryo, (ii) prolonged undifferentiated proliferation, and (iii) stable developmental potential to form derivatives of all three embryonic germ layers (endoderm, mesoderm and ectoderm) even after prolonged culture.

For human embryonic stem cells, it has been demonstrated that these cells may be obtained from frozen-thawed blastocysts that were destined to be discarded after 5 years in a routine human IVF-embryo transfer programme (Park et al., 2004). ES cells grow as homogenous and undifferentiated colonies when they are propagated on a feeder layer such as mouse embryonic fibroblasts. Removal from this feeder layer is associated with differentiation into derivatives of the three embryonic germ layers. Human embryonic stem cells have been described to be able to differentiate in vitro into pancreatic endocrine cells, and particularly into β cells (Assady et al., 2001).

In a particular embodiment, pluripotent stem cells are derived from non-human embryonic stem cells.

Stem cells as described above, which have the capacity to differentiate into pancreatic endocrine cells and thus into their precursors, namely Ngn3+ endocrine progenitor cells, may be used in the method of the invention for increasing the pool of these Ngn3+ cells.

The step of contacting with a CFTR inhibitor or an inhibitor of CFTR gene expression has to be conducted after detection of pdx1 gene expression and before the complete differentiation of these cells into pancreatic endocrine cells, preferably after detection of pdx1 gene expression and before detection of Ngn3 expression.

Stem cells as described above may be derived from any mammalian such as mice, rats, pigs, dogs, cats, horses, monkeys or humans.

In the present method, stem cells having the capacity to differentiate into pancreatic endocrine cells are contacted with a CFTR inhibitor or an inhibitor of CFTR gene expression. This step of contacting stem cells with a CFTR inhibitor or an inhibitor of CFTR gene expression may consist of culturing stem cells in a medium containing a CFTR inhibitor or an inhibitor of CFTR gene expression.

The concentration of the CFTR inhibitor or inhibitor of CFTR gene expression may be chosen by the skilled person using well known methods. For instance, preliminary tests may be achieved to evaluate the toxicity of the CFTR inhibitor or inhibitor of CFTR gene expression on stem cells. In this case, stem cells are cultured with different concentrations of this inhibitor and toxicity markers are followed. These markers may be markers of apoptotic cell death such as apoptotic DNA fragmentation and DEVD-caspase activation. The concentration of the inhibitor has to be chosen in order to be safe of any toxic effects on growing stem cells. Preferably, the concentration is chosen in order to be the highest concentration without any toxic effect.

The culture medium which may be used during the step of contacting with a CFTR inhibitor or inhibitor of CFTR gene expression is designed to support the growth and the differentiation of stem cells. This medium generally is changed every day and comprises a carbon source, a nitrogen source, antibiotics to prevent fungi and bacteria growth, a buffer to maintain pH and specific growth factors. This medium may be easily designed by the skilled person in the art. An example of such medium is presented in the experimental section below or in the experimental section of the article of Guillemain et al (Guillemain et al., 2007).

Other compounds may also be added in the medium such as compound known to stimulate β cell replication, to induce differentiation into β cells or to inhibit apoptosis of β cells. Such compounds may be chosen from the group consisting of nicotinamide, glucagon-like peptide-1 (GLP-1), glucose, exendin-4 and retinoic acid.

In an embodiment, stem cells are contacted with a CFTR inhibitor or an inhibitor of CFTR gene expression during 3 to 10 days, preferably from 5 to 7 days. During the step of contacting, stem cells are cultured in a medium supporting growth and differentiation and containing a CFTR inhibitor or inhibitor of CFTR gene expression.

At the end of the step of contacting and/or several days later, the number of Ngn3-expressing cells may be assessed in order to verify the efficiency of the treatment, i.e. the increase of the pool of Ngn3+ endocrine progenitor cells. The number of Ngn3-expressing cells obtained in treated samples is compared to the number of Ngn3-expressing cells obtained in control sample, i.e. without step of contacting with a CFTR inhibitor or an inhibitor of CFTR gene expression. In order to be comparable, stem cells in treated and control samples have to be of the same cellular type and submitted to the same protocol except channel inhibitor treatment.

In an embodiment, the pool of Ngn3+ endocrine progenitor cells has increased by more than 25%, preferably by more than 50% and the most preferably by more than 100%.

In a preferred embodiment, the pool of Ngn3+ endocrine progenitor cells has increased by more than 150%, preferably by more than 200% and the most preferably by more than 250%.

In an embodiment, the method further comprises a step consisting of the differentiation of obtained Ngn3+ endocrine progenitor cells into precursors of pancreatic endocrine cells and/or pancreatic endocrine cells.

In appropriate culture medium, such as described above, Ngn3+ endocrine progenitor cells differentiate into precursors of pancreatic endocrine cells and subsequently into β, δ, e and/or PP cells.

The present invention also concerns an in vitro method for increasing the number of pancreatic endocrine cells obtained from stem cells, wherein said method comprises the step of contacting stem cells having the capacity to differentiate into pancreatic endocrine cells with a CFTR inhibitor or an inhibitor of CFTR gene expression.

The present invention also concerns an in vitro method for increasing the β cell mass obtained from stem cells, wherein said method comprises the step of contacting stem cells having the capacity to differentiate into pancreatic endocrine cells with a CFTR inhibitor or inhibitor of CFTR gene expression.

In a particular embodiment, this method comprises the step of contacting stem cells having the capacity to differentiate into pancreatic endocrine cells, except human embryonic stem cells, due to some patent law and practices.

The present invention also relates to an in vitro method for obtaining pancreatic endocrine cells, wherein said method comprises the step of contacting stem cells having the capacity to differentiate into pancreatic endocrine cells with a CFTR inhibitor or an inhibitor of CFTR gene expression.

In another aspect, the present invention also provides an in vivo method for increasing the number of pancreatic endocrine cells, in particular of β cells, in the pancreas of a foetus, wherein said method comprises administering a CFTR inhibitor or an inhibitor of CFTR gene expression to the pregnant female.

In another aspect, the present invention also provides an in vivo method for increasing the number of pancreatic endocrine cells, in particular of β cells, in the pancreas of a subject, wherein said method comprises administering a CFTR inhibitor or an inhibitor of CFTR gene expression to said subject. Preferably, the subject is a child.

In a further aspect, the present invention provides pancreatic cells obtained by the in vitro method of the invention.

In an embodiment, pancreatic cells are Ngn3+ endocrine progenitor cells.

In another embodiment, pancreatic cells are cells derived from Ngn3+ endocrine progenitor cells, i.e. pancreatic endocrine cell precursors and pancreatic endocrine cells.

Precursors of pancreatic endocrine cells may express, for instance, Pax4 (paired boxencoding gene 4) or Arx (Aristaless-related homeobox). Pancreatic endocrine cells may be α, β, δ, e and/or PP cells.

In a preferred embodiment, pancreatic cells are β cells. The term “beta cells”, as used herein, refers to pancreatic cells which are able to produce insulin. In vivo, these cells are found in the pancreatic islets of Langerhans. This cell population may be identified by the expression of specific markers such as ZnT-8, a specific zinc transporter (Chimienti et al. 2004) or MafA, a specific transcription factor (Zhang et al., 2005; Matsuoka et al., 2007), or by an ability to respond to glucose challenge in a specific way by secreting insulin.

The present invention also relates to pancreatic islets comprising pancreatic cells of the invention as described above.

As used herein, the term “pancreatic islet” refers to cell small discrete cell aggregates obtained in vitro or ex vivo and including pancreatic endocrine hormone producing cells, such as α cells, β cells, δ cells, PP cells and e cells. Pancreatic islets resemble the form of islets of Langerhans of the pancreas and are spheroid in form. In vivo, the islets of Langerhans are surrounded by the pancreatic exocrine tissue.

In an embodiment, pancreatic islets comprise β cells obtained by the method of the invention.

In another embodiment, pancreatic islets comprise β cells and α cells obtained by the method of the invention.

In a preferred embodiment, pancreatic islets comprise α cells, β cells, δ cells, PP cells and e cells obtained by the method of the invention.

In another aspect, the present invention concerns pancreatic cells and/or pancreatic islets of the invention for the treatment of diabetes in a subject in need thereof.

The present invention also relates to a method of treating disorders of glucose homeostasis in a subject in need thereof, said method comprising steps consisting of
obtaining stem cells having the capacity to differentiate into pancreatic endocrine cells;
contacting said stem cells with a CFTR inhibitor or an inhibitor of CFTR gene expression during their differentiation into pancreatic endocrine cells;
transplanting a therapeutically effective amount of pancreatic islets obtained by differentiation of said stem cells into said subject.

Once transplanted, the pancreatic islets begin to produce insulin, actively regulating the level of glucose in the blood. The main obstacle in islet transplantation is the fact that there is an inadequate supply of cadaveric islets to implement this procedure on a widespread clinical basis. The method of the invention solves this problem by obtaining an increase number of pancreatic islets which may be used for transplantation.

Typically, it was estimated that a diabetic patient needs at least 10,000 pancreatic islets per kilogram body weight to achieve a measurable increase in insulin production. Generally, between 10,000 and 30,000 pancreatic islets per kilogram body weight are administered to the subject during transplantation. The number of pancreatic islets to be administered to a subject will vary depending on a number of parameters including the size of the subject, the severity of the disease and the site of implantation.

Generally pancreatic islets are suspended in a pharmacologically acceptable carrier, such as, for instance, cell culture medium (such as Eagle's minimal essential media), phosphate buffered saline, Krebs-Ringer buffer, and Hank's balanced salt solution +/−glucose (HBSS).

The pancreatic islets can be administered by any method known to one of skill in the art.

In an embodiment, pancreatic islets are administered by injection. For example, pancreatic islets may be administered by subcutaneous injection, intra-peritoneal injection, injection under the kidney capsule, injection through the portal vein and injection into the spleen.

According to the origin of stem cells, the islet transplantation may be autologous, isogeneic, allogeneic or xenogeneic. As used below, the “donor” is the donor of stem cells and the “recipient” is the subject who receives the islet transplantation. In an embodiment, the islet transplantation is isogeneic, i.e. the donor and recipient are genetically identical. In another embodiment, the islet transplantation is allogeneic, i.e. the donor and recipient are of the same species. In another embodiment, the islet transplantation is xenogeneic, i.e. the donor and recipient are of different species. Allogeneic and xenogeneic transplantation require the administration of antirejection drugs. For isogeneic, allogeneic and xenogeneic transplantation, the donor may be alive or deceased. In a preferred embodiment, the islet transplantation is autologous, i.e. the donor and recipient are the same subject. In this case, stem cells may be (i) derived from adult tissue of the subject, (ii) derived from somatic cells of said subject which have been reprogrammed to provide induced pluripotent stem cells or (iii) from embryonic stem cells obtained by cloning.

### Pharmaceutical Compositions and Implants

The active ingredients of the invention may be combined with pharmaceutically acceptable excipients, and optionally sustained-release matrices, such as biodegradable polymers, to form therapeutic compositions.

As used herein, the term “active ingredient of the invention” is intended to refer to the CFTR inhibitors, inhibitors of CFTR gene expression, progenitor cells, pancreatic endocrine cells and pancreatic islets as defined above.

Accordingly, a further aspect of the invention relates to a pharmaceutical composition comprising an active ingredient of the invention for use in the treatment of disorders of glucose homeostasis.

The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention, the active ingredients of the invention can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The active ingredients of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active ingredients of the invention in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

In addition to the active ingredients of the invention formulated for parenteral administration, such as intravenous or intramuscular injection, other pharmaceutically acceptable forms include, e.g. tablets or other solids for oral administration; liposomal formulations; time release capsules; and any other form currently used.

The present invention also relates to a biodegradable implant comprising an active ingredient of the invention for use in the treatment of disorders of glucose homeostasis.

The implants can be formed in manner that the active ingredient is homogenously distributed or dispersed throughout the biodegradable polymer matrix. Additionally, the implants can be formed to release the active ingredient into an pancreatic region of the pancreas over various time periods. Thus, the active ingredient can be released from implants made according to the present invention for a period of time of, for example, 30-40 days.

The active ingredient can comprise from about 10% to about 90% by weight of the implant. In one variation, the agent is from about 40% to about 80% by weight of the implant. In a preferred variation, the agent comprises about 60% by weight of the implant

In a particular embodiment, the active ingredient can be homogeneously dispersed in the biodegradable polymer of the implant. The implant can be made, for example, by a sequential or double extrusion method. The selection of the biodegradable polymer used can vary with the desired release kinetics, patient tolerance, the nature of the disease to be treated, and the like. Polymer characteristics that are considered include, but are not limited to, the biocompatibility and biodegradability at the site of implantation, compatibility with the active ingredient of interest, and processing temperatures. The biodegradable polymer matrix usually comprises at least about 10, at least about 20, at least about 30, at least about 40, at least about 50, at least about 60, at least about 70, at least about 80, or at least about 90 weight percent of the implant. In one variation, the biodegradable polymer matrix comprises about 40% to 50% by weight of the implant.

Biodegradable polymers which can be used include, but are not limited to, polymers made of monomers such as organic esters or ethers, which when degraded result in physiologically acceptable degradation products Anhydrides, amides, orthoesters, or the like, by themselves or in combination with other monomers, may also be used. The polymers are generally condensation polymers. The polymers can be crosslinked or non-crosslinked. If crosslinked, they are usually not more than lightly crosslinked, and are less than 5% crosslinked, usually less than 1% crosslinked. Of particular interest are polymers of hydroxyaliphatic carboxylic acids, either homo- or copolymers, and polysaccharides. Included among the polyesters of interest are homo- or copolymers of D-lactic acid, L-lactic acid, racemic lactic acid, glycolic acid, caprolactone, and combinations thereof. Copolymers of glycolic and lactic acid are of particular interest, where the rate of biodegradation is controlled by the ratio of glycolic to lactic acid. The percent of each monomer in poly(lactic-co-glycolic)acid (PLGA) copolymer may be 0-100%, about 15-85%, about 25-75%, or about 35-65%. In certain variations, 25/75 PLGA and/or 50/50 PLGA copolymers are used. In other variations, PLGA copolymers are used in conjunction with polylactide polymers.

Other agents may be employed in the formulation for a variety of purposes. For example, buffering agents and preservatives may be employed. Preservatives which may be used include, but are not limited to, sodium bisulfite, sodium bisulfate, sodium thiosulfate, benzalkonium chloride, chlorobutanol, thimerosal, phenylmercuric acetate, phenylmercuric nitrate, methylparaben, polyvinyl alcohol and phenylethyl alcohol. Examples of buffering agents that may be employed include, but are not limited to, sodium carbonate, sodium borate, sodium phosphate, sodium acetate, sodium bicarbonate, and the like, as approved by the FDA for the desired route of administration. Electrolytes such as sodium chloride and potassium chloride may also be included in the formulation.

The biodegradable pancreatic implants can also include additional hydrophilic or hydrophobic compounds that accelerate or retard release of the active ingredient. Additionally, release modulators such as those described in U.S. Pat. No. 5,869,079 can be included in the implants. The amount of release modulator employed will be dependent on the desired release profile, the activity of the modulator, and on the release profile of the active ingredient in the absence of modulator. Where the buffering agent or release enhancer or modulator is hydrophilic, it may also act as a release accelerator. Hydrophilic additives act to increase the release rates through faster dissolution of the material surrounding the drug particles, which increases the surface area of the drug exposed, thereby increasing the rate of drug diffusion. Similarly, a hydrophobic buffering agent or enhancer or modulator can dissolve more slowly, slowing the exposure of drug particles, and thereby slowing the rate of drug diffusion.

The release kinetics of the implants of the present invention can be dependent in part on the surface area of the implants. A larger surface area exposes more polymer and active ingredient to pancreatic fluid, causing faster erosion of the polymer matrix and dissolution of the active ingredient particles in the fluid. Therefore, the size and shape of the implant may also be used to control the rate of release, period of treatment, and active ingredient concentration at the site of implantation. At equal active ingredient loads, larger implants will deliver a proportionately larger dose, but depending on the surface to mass ratio, may possess a slower release rate. For implantation in an pancreatic region, the total weight of the implant preferably ranges, e.g., from about 200-15000 [mu]g, usually from about 1000-5000 [mu]g. In one variation, the total weight of the implant is about 1200 to about 1,800 [mu]g. In another variation, the total weight of the implant is about 2400 to about 3,600 [mu]g. Preferably, the implant has a weight between about 100 [mu]g and about 2 mg.

The implants of the invention are typically solid, and may be formed as particles, sheets, patches, plaques, films, discs, fibers, rods, and the like, or may be of any size or shape compatible with the selected site of implantation, as long as the implants have the desired release kinetics and deliver an amount of active ingredient that is therapeutic for the intended medical condition of the pancreas. The upper limit for the implant size will be determined by factors such as the desired release kinetics, toleration for the implant at the site of implantation, size limitations on insertion, and ease of handling. For example, the vitreous chamber is able to accommodate relatively large rod-shaped implants, generally having diameters of about 0.05 mm to 3 mm and a length of about 0.5 to about 10 mm. In one variation, the rods have diameters of about 0.1 mm to about 1 mm. In another variation, the rods have diameters of about 0.3 mm to about 0.75 mm. In yet a further variation, other implants having variable geometries but approximately similar volumes may also be used.

### Diagnostic Methods

A further aspect of the invention relates to a method of testing a subject thought to have or be predisposed to having disorders of glucose homeostasis, which comprises the step of analyzing a sample of interest from said subject for:

(i) detecting the presence of a mutation in the CFTR gene and/or its associated promoter, and/or

(ii) analyzing the expression of the CFTR gene.

As used herein, the term “sample of interest” include encompasses a variety of sample types obtained from a subject and can be used in a diagnostic assay. Samples herein may be any type of sample, such as any cell samples (e.g. pancreatic cells), biological fluids including, blood, serum, urine, spinal fluid . . . or any biopsy sample obtained from a subject's tissue (e.g. pancreas).

Without wishing to be bound by theory, the inventors believe that aberrant expression or activity of CFTR, and/or especially its ability to cAMP-mediated chloride secretion may determine whether an individual is afflicted with disorders of glucose homeostasis, or is at risk of developing disorders of glucose homeostasis. For example, certain polymorphisms in the CFTR gene may render the protein constitutively active or more active and thus may limit the increasing of the NGN3+ endocrine progenitor cells and finally the final number of beta cells that develop.

Typical techniques for detecting a mutation in the CFTR gene may include restriction fragment length polymorphism, hybridisation techniques, DNA sequencing, exonuclease resistance, microsequencing, solid phase extension using ddNTPs, extension in solution using ddNTPs, oligonucleotide assays, methods for detecting single nucleotide polymorphism such as dynamic allele-specific hybridisation, ligation chain reaction, mini-sequencing, DNA “chips”, allele-specific oligonucleotide hybridisation with single or dual-labelled probes merged with PCR or with molecular beacons, and others.

Analyzing the expression of the CFTR gene may be assessed by any of a wide variety of well-known methods for detecting expression of a transcribed nucleic acid or translated protein.

In a preferred embodiment, the expression of the CFTR gene is assessed by analyzing the expression of mRNA transcript or mRNA precursors, such as nascent RNA, of said gene. Said analysis can be assessed by preparing mRNA/cDNA from cells in a sample of interest from a subject, and hybridizing the mRNA/cDNA with a reference polynucleotide. The prepared mRNA/cDNA can be used in hybridization or amplification assays that include, but are not limited to, Southern or Northern analyses, polymerase chain reaction analyses, such as quantitative PCR (TaqMan), and probes arrays such as GeneChip™ DNA Arrays (AFF YMETRIX).

Advantageously, the analysis of the expression level of mRNA transcribed from the CFTR gene involves the process of nucleic acid amplification, e.g., by RT-PCR (the experimental embodiment set forth in U.S. Pat. No. 4,683,202), ligase chain reaction (BARANY, Proc. Natl. Acad. Sci. USA, vol. 88, p: 189-193, 1991), self sustained sequence replication (GUATELLI et al., Proc. Natl. Acad. Sci. USA, vol. 57, p: 1874-1878, 1990), transcriptional amplification system (KWOH et al., 1989, Proc. Natl. Acad. Sci. USA, vol. 86, p: 1173-1177, 1989), Q-Beta Replicase (LIZARDI et al., Biol. Technology, vol. 6, p: 1197, 1988), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

In another preferred embodiment, the expression of the CFTR gene is assessed by analyzing the expression of the protein translated from said gene. Said analysis can be assessed using an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from the CFTR gene.

Said analysis can be assessed by a variety of techniques well known from one of skill in the art including, but not limited to, enzyme immunoassay (EIA), radioimmunoassay (RIA), Western blot analysis and enzyme linked immunoabsorbant assay (RIA).

The method of the invention may comprise comparing the level of expression of the CFTR gene in a sample of interest from a subject with the normal expression level of said gene in a control. A significantly higher level of expression of said gene in the sample of interest of a subject as compared to the normal expression level is an indication that the patient has or is predisposed to developing disorders of glucose homeostasis. The “normal” level of expression of the CFTR gene is the level of expression of said gene in a sample of interest of a subject not afflicted by any disorders of glucose homeostasis. Preferably, said normal level of expression is assessed in a control sample (e.g., sample from a healthy subject, which is not afflicted by any disorders of glucose homeostasis) and preferably, the average expression level of said gene in several control samples.

The invention will be further illustrated by the following figures and examples. However, these examples and figures should not be interpreted in any way as limiting the scope of the present invention.

### FIGURES

FIG. 1: Effects of GlyH-H101 on endocrine cell development. Quantification by real-time PCR of insulin (A) and MafA (B) mRNA transcripts in E 13.5 Rat pancreases before (D0) and after 1, 3, 5 and 7 days of culture (D1, D3, D5 and D7 respectively) with or without 10 μM GlyH-101. Each data point represents the mean±sem of at least four independent experiments ***, p<0.001. (C) Immunohistological analysis of E13.5 Rat pancreases cultured 5 days in the presence or in absence of 10 μM GlyH-101. Insulin and amylase staining were respectively in red and green. Nuclei were stained in blue with Hoechst. Scale bar: 50 μm (D). β cell differentiation was evaluated by quantification of the absolute surface area occupied by insulin staining in E 13.5 Rat pancreases after 5 days of culture in presence or in absence of 10 μM GlyH-101. Values are means±sem of at least three independent experiments *, p<0.05; (E) Quantification by real-time PCR of Ngn3 transcripts in E13.5 Rat pancreases before (D0) and after 1, 3, 5 and 7 days of culture (D0, D1, D3, D5 and D7 respectively) with or without 10 μM GlyH-101. Each data point represents the mean±sem of at least three independent experiments **, p<0.01; ***, p<0.001. (F) Immunohistochemistry analysis of NGN3 expression in E13.5 Rat pancreases cultured 5 days in presence or in absence of 10 μM GlyH-101. Note the increased number of NGN3 positive nuclei in the GlyH-101-treated explants. Scale bar: 50 μm. (G) Quantification of the number of NGN3 positive-cells in E13.5 Rat pancreases cultured for 5 days in presence or in absence of 10 μM GlyH-101. Values are means±sem of at least three independent experiments **, p<0.01. Quantification of the surface area occupied by insulin expressing cells that developed in E12.5 pancreases from WT (CFTR+/+)(H) and knockout (CFTR−/−) (I) mice cultured for 6 days with or without 10 μM GlyH-101. Data are presented as means±sem for at least five mice per group (*, p<0.05).

### Example 1

### Material & Methods

Animals and Pancreatic Dissection:

Pregnant Wistar rats were purchased from the Janvier Breeding centre (CERJ, LeGenet, France). Cftr-knockout (Cftr−/−) and wild-type (Cftr+/+) embryos were obtained from the progeny of heterozygous-heterozygous for the S489X mutation (Snouwaert et al. 1992) matings (CDTA, Orléans, France). The first day postcoitum was taken as embryonic day (E)0.5. Pregnant rats were killed with CO₂ asphyxiation and pregnant mice by cervical dislocation according to guidelines issued by the French Animal Care Committee. Dorsal pancreatic buds from E13.5 rat and E12.5 mice embryos were dissected as described previously (Miralles et al. 1998). Briefly, the stomach, pancreas and a small portion of the intestine were dissected together, and then the pancreas primordium was isolated.

Organ Culture:

Dorsal pancreatic rudiments were cultured on 0.45 μm filters (Millipore) at the air-medium interface in a 35 mm sterile Petri dishes containing 2 ml RPMI-1640 medium (Invitrogen) supplemented with 100 U/ml penicillin, 100 μg/ml streptomycin, 10 mmol/L HEPES, 2 mmol/L L-glutamine, 1× non-essential aminoacids (Invitrogen) and 10% heat-inactivated fetal calf serum (HyClone, Logan, Utah, USA). Glibenclamide (MP Biomedical), and Glycine hydrazide (GlyH-101)(Calbiochem), used at the indicated concentrations, were first dissolved as concentrated solutions in dimethylsulfoxide DMSO (Sigma), the final concentration of DMSO in the culture medium was less than 0.5% (vol/vol). Glibenclamide and GlyH-101 were added to the media daily. The cultures were incubated at 37° C. in a humidified atmosphere composed of 95% air and 5% CO₂. At the end of the culture period, the pancreases were photographied, and fixed as described below or harvested for RNA extraction.

Immunochemistry and Surface Quantification:

Immunochemistry—

The pancreatic rudiments were fixed in 10% formalin, pre-embedded in agarose gel (4% of type VII low-gelling-temperature agarose [Sigma] in H₂O) and embedded in paraffin. Immunohistochemistry was performed on 4-μm paraffin sections as previously described) (Duvillie et al. 2006). The primary antibodies were mouse anti insulin (1/2000; Sigma), rabbit anti-glucagon (1/1000; Diasorin), rabbit anti-amylase (1/300; Sigma), rabbit anti-carboxypeptidase A (CPA) (1/600; Biogenesis, Kidlington, Oxford, UK), rabbit anti-PDX1 (1/1000) (Duvillie et al. 2006) mouse anti-BrdU (1/2; Amersham Biosciences, Buckingham, UK), rabbit anti-Proprotein Convertase Subtilisin/Kexin 1/3 (PCSK1/3), rabbit anti-Ngn3 (1/100, (Guillemain et al. 2007) The fluorescent secondary antibodies were fluorescein anti-rabbit antibody (1/200; Jackson Immunoresearch, Baltimore, Md., USA), fluorescein goat anti-rabbit Alexa Fluor 488 (1/400; Invitrogen) and Texas red anti-mouse antibody (1/200; Jackson). Nuclei were stained in blue with Hoechst 33342 (0.3 μg/ml; Invitrogen). Ngn3 detection was performed as previously described (Guillemain et al. 2007) using the Vectastain elite ABC kit (Vector laboratories).

Photographs were taken using a fluorescence microscope (Leica, Leitz DMRB, Reuil-Malmaison, France) and digitized using a Hamamatsu (Middlesex, N.J.) C5810 cooled 3CCD camera.

Quantification—

To quantify the surface area of insulin, glucagon, PCSK1/3, CPA and amylase-expressing cells, all sections of each pancreatic rudiment were digitized. Alternate sections were examined to avoid counting the same cell twice. The surface of insulin, glucagon, PCSK1/3, CPA, amylase and Hoechst stainings were quantified using Iplab (Scanalytics). The stained areas were summed to obtain the total surface area per rudiment in mm². To measure proliferation of the early progenitors expressing PDX1, we counted the frequency of BrdU positive progenitors expressing PDX1 among 3000 early progenitors expressing PDX1 per rudiment. To quantify the absolute number of NGN3-expressing cells, pancreatic rudiments were sectioned and all sections were stained with an anti-NGN3 antibody. Positive cells were counted on all sections of each pancreatic rudiment. A minimum of three rudiments was analyzed per condition.

RNA Extraction and Real-Time PCR:

Total RNA was isolated from pools of at least three pancreases using the Qiagen RNeasy Microkit (Qiagen, Courtaboeuf, France) and reverse transcribed using Superscript reagents (Invitrogen). Real-time PCR was performed with the 7300 Fast real-time PCR system (Applied Biosystem) using either Taqman universal PCR master mix or SYBR green PCR master mix (Applied Bio system) with primers and labelled probes specific for each gene. Peptidylpropyl isomerase A/Cyclophilin A was used as endogenous control and E16.5 pancreas cDNA as calibrator sample. The data were analyzed by comparative cycle threshold method (Livak and Schmittgen 2001) and presented as the fold change in gene expression. At least three pools of explants were analysed by condition.

Statistical Analysis:

All results are expressed as mean±sem. Statitical significance was determined using Student's t test.

Results

High Concentrations of Glibenclamide Did not Alter the Morphology of the Developing Pancreas In Vitro:

Using our in vitro model, E13.5 Rat embryonic pancreases cultured at the air/medium interface on a floating filter, we examined the effects of increased concentrations of the sulphonylurea glibenclamide (an inhibitor of K_{ATP} channels) on pancreas development. The pancreatic growth was similar in absence (control) or in presence of 10 nM, 100 nM, 1, 10 or 100 μM glibenclamide during the 7 days of culture. Under both conditions, the epithelium grew rapidly, spread into the mesenchyme and developed lobules. There is no difference in apoptosis in pancreases cultured 7 days without or with 10 and 100 μM glibenclamide as shown by the Hoechst staining of the nuclei. Moreover, the lack of glibenclamide toxicity on the developing pancreas was confirmed by the quantitative analysis of the overall size of pancreases cultured 7 days in the presence or in the absence of 10 or 100 μM glibenclamide.

Based on these first results, in particular the lack of glibenclamide toxicity on the pancreas morphology and on the effects on the pro-endocrine progenitor cells, the 100 μM glibenclamide concentration was used in the next experiments.

Glibenclamide Did not Modify the Pancreatic Exocrine Development:

To evaluate the effects of glibenclamide treatment on exocrine development, we cultured for 7 days E13.5 pancreases in presence of 100 μM glibenclamide and assessed the expression of the acinar (amylase, carboxypeptidaseA) and ductal tissues differentiation markers (CFTR, Cystic Fibrosis Transmembrane conductance Regulator- and osteopontin-SPP1) (Hyde et al. 1997; Kilic et al. 2006). No differences were found in the surface area occupied by amylase- and carboxypeptidaseA-positive cells after 7 days of culture in presence or in absence of glibenclamide. This result was further confirmed with the amylase expression profile during the 7 days of culture. Analysis of the expression pattern of SPP1 and CFTR before and after 1, 3, 5 and 7 days of culture. These findings indicate that glibenclamide did not to modify the exocrine (acinar and ductal) development.

Effects of Glibenclamide on β and α Cells Differentiation:

To determine the effects of glibenclamide on endocrine development and in particular in cells expressing SUR1, we first compared insulin-positive cells, in pancreases grown for 7 days in the absence or the presence of glibenclamide. The glibenclamide treated-explants exhibit a very few insulin positive cells and the surface area occupied by the insulin-cell population was decreased by 70%. Because glibenclamide is a potent insulin secretagogue, we asked if the observed low insulin content was only the consequence of increased insulin secretion in the culture medium or was due to a reduction of insulin mRNA level. To test this latter hypothesis, we analysed by real-time PCR the expression of insulin gene before (D0) and after 1, 3, 5 and 7 days of culture. The insulin expression was strongly reduced in the glibenclamide-treated pancreases as early as D3. Two mechanisms can account for the observed decrease in insulin expression: (i) inhibition of β-cell differentiation from the pro-endocrine progenitors leading to a reduction of the β-cell number and thus to a decrease in overall amount of insulin mRNA or (ii) inhibition of the insulin gene without affecting the β cell population. Thus, we examined the expression pattern of two β-cell markers: the zinc transporter ZnT-8 (Chimienti et al. 2004) and the β-cell specific transcription factor MafA (Zhang et al. 2005). Our results indicate that over the 7 days of culture, glibenclamide did not affect the expression of these two beta-cells markers. Moreover, after 7 days of culture, the surface area occupied by the pro-hormone convertase PCSK1/3 staining in PDX-1⁺ cells ((Pdx-1 is specifically expressed in the adult β-cell (Ohlsson et al. 1993)) was similar in pancreases cultured without or with glibenclamide. These results strongly suggest that glibenclamide did not prevent the β-cell differentiation.

In contrast to the lack of effects on β-cell development, glibenclamide increased by 3.5 fold the number of glucagon-expressing cells. Moreover, this result was confirmed by the significant increase of Pou3F4/Brn4 mRNA level at D7; Pou3F4 is the only α-cell specific transcription factor which maintains the α-cell fate (Heller et al. 2004). In the other hand, we found also a twofold increase in somatostatin expression after 7 days of culture in the glibenclamide-treated pancreas. Taken together, our results demonstrate that glibenclamide did not prevent the differentiation of β-cells, but increase the α-cell number.

Glibenclamide Amplifies the Pool of Endocrine Progenitors, Increases the Expression of the Ngn3 Target but does not Affect the Proliferation of Pancreatic Precursors:

The pancreatic endocrine fate is determined by the expression of Ngn3, a transcription factor which specifically labels the endocrine precursors (Gradwohl et al. 2000; Gu et al. 2002). Thus, we asked if the increase of α-cell number was due to an increase of NGN3+ pro-endocrine cells. To test this hypothesis, we investigated the expression pattern of Ngn3 before and after 1, 3, 5 and 7 days of culture. Ngn3 was weakly expressed at E13.5 (D0). It increases at day 1 and 3 but remain similar in absence or in presence of glibenclamide. In contrast, after 5 days of culture, Ngn3 expression reached a peak and was sevenfold increased by glibenclamide (p<0.001). Thereafter, Ngn3 mRNA level decreased slightly but remained dramatically higher (p<0.001) in the glibenclamide-treated pancreases. In the next step, we asked whether glibenclamide acts not only on Ngn3 gene expression but also on NGN3-expressing cell number. Thus, NGN3 expression was analysed by immunohistochemistry and the number of NGN3+ cells was compared in pancreases cultured for 5 days in absence or in presence of glibenclamide. The number of NGN3+ cells that develop in presence of glibenclamide was threefold higher than in absence of glibenclamide. These results suggest strongly that glibenclamide amplifies the pool of NGN3+ endocrine progenitors. Because the transcription factor NeuroD1/Beta2 is a downstream target of Ngn3 (Huang et al. 2000) and is necessary to the endocrine differentiation (Guillemain et al. 2007)-we examined the expression pattern of this Ngn3 target. mRNA levels were similar after 1 and 3 days of culture in absence or in presence of glibenclamide. In concordance with the expression pattern of Ngn3 in the glibenclamide-treated pancreases, NeuroD1 was significantly increased at D5 and reminded enhanced at D7. These results suggest strongly that the overexpression of Ngn3 in our in vitro model leads to the induction of a key factor important for islet differentiation.

We thus asked whether glibenclamide increased NGN3+ cell number by acting on pancreatic progenitor cell proliferation. We cultured embryonic pancreases for 1 day and added BrdU during the last hour of culture. The percentage of PDX-1+ cells that incorporate BrdU was similar in presence (32.60%±4.2%) or in absence (31.24%±2.8%) of glibenclamide. Thus, glibenclamide did not modify pancreatic precursor proliferation.

In conclusion, glibenclamide amplifies the pool of pro-endocrine cells expressing Ngn3 without acting on the proliferation of pancreatic progenitors.

NGN3+ Cells Induced by Glibenclamide Differentiate into Beta Cells:

We showed a dramatic increase of Ngn3 expression, with a peak at D5 in glibenclamide-treated pancreases (sevenfold when compared to the control). As (i) β cells develop from NGN3+ expressing cells (ii) no effect of glibenclamide on beta cell differentiation was observed at D7, we thus asked whether the pro-endocrine NGN3+ cells induced by glibenclamide could differentiate into beta cells after a 7-day culture period. To answer this question, we cultured pancreases up to 14 days in presence of glibenclamide only during the first 5 days of culture, i.e until Ngn3 reaches a peak (Glib-5D pancreases), in absence (Control pancreases) or in presence of glibenclamide (Glib pancreases) during the 14-day culture period. Then, we compared insulin-expressing cells that developed in the absence of glibenclamide with the one that developed in Glib-5D and Glib treated pancreases after 9, 11 and 14 days of culture. A large number of insulin-expressing cells were observed in Glib-5D pancreases at D 9, D11 or D14. In contrast, less insulin-containing cells were detected in the pancreases cultured 9, 11 or 14 days in presence of glibenclamide. Such an inhibitory effect of glibenclamide on insulin expression and content without affecting the beta cell number has been already mentioned above.

By real real-time PCR, we assessed the insulin expression after 7, 9, 11 and 14 days of culture. As shown in FIG. 5-C, while insulin mRNA level was identical at D7, it increased by threefold at D9, twofold at D11 and threefold at D14 in pancreases cultured with glibenclamide the first 5 days of culture (Glib 5D), when compared with those cultured without glibenclamide. This result was further confirmed by the quantification of insulin-staining area which shows a significant increase of insulin-positive cells in the Glib 5D-treated pancreas.

To check that the strong activation of insulin expression, along with the increase of insulin-positive cells observed in Glib 5D-treated pancreas, were correlated to an increase of β-cell differentiation, we analysed the expression pattern of the two beta-cell markers ZnT-8 and MafA at D7, D9, D11 and D14. Whereas the ZnT-8 and MafA expression were similar after 7 days of culture, a dramatic increase (p<0.01) of these two β-cell markers expression was observed after 9, 11 and 14 days of culture in the Glib 5D-treated pancreas suggesting that β-cell number is increased by glibenclamide treatment.

On the other hand, the fact that the β-cell differentiation occurred after 9, 11 and 14 days of culture on pancreas treated with glibenclamide only during the first 5 days suggests that glibenclamide activate β-cell development by acting upstream of Ngn3.

The main conclusion of these findings is that glibenclamide-induced NGN3+ cells have the ability to differentiate in β-cells.

Inhibition of CFTR Channel Mimics Glibenclamide Effects on Endocrine Progenitor NGN3⁺ Cells:

Glibenclamide was shown to block CFTR chloride channel in mammalian cardiac myocytes (Yamazaki and Hume 1997), 3T3 fibroblasts (Sheppard and Welsh 1992) or mammary murine cell line expressing recombinant human CFTR (Sheppard and Robinson 1997). Glibenclamide was also reported to inhibits CFTR channel in a dose-dependant manner via an open-channel block mechanism (Schultz et al. 1996). To test the hypothesis that glibenclamide effects observed on pancreas development and in a particular on the endocrine lineage could be mediated by the closure of CFTR channel, we grow E13.5 Rat pancreases with 10 μM GlyH-101, a specific inhibitor of CFTR (Muanprasat et al. 2004) for up to 7 days.

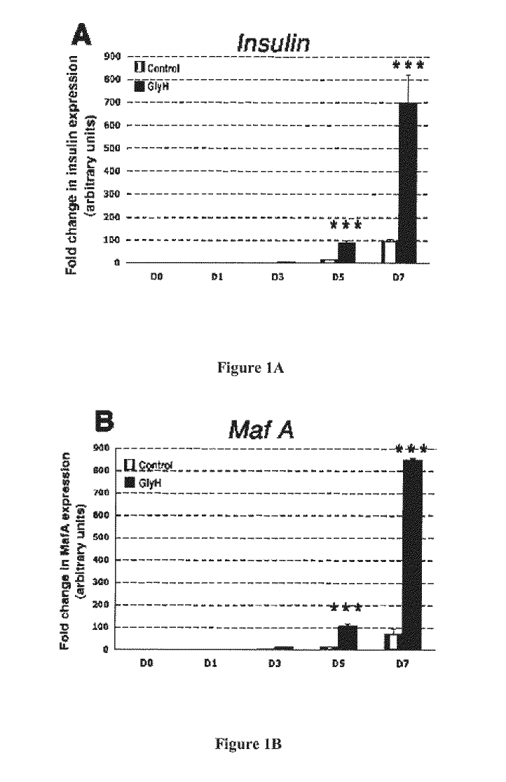

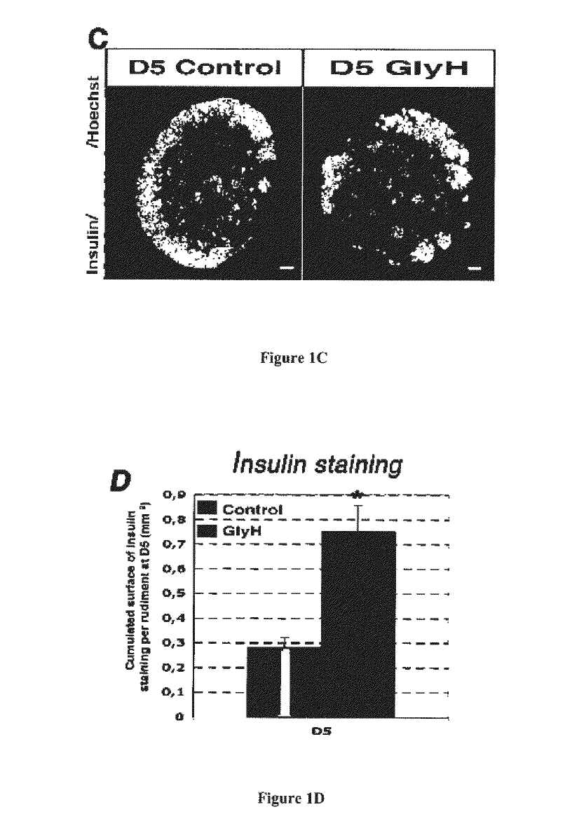

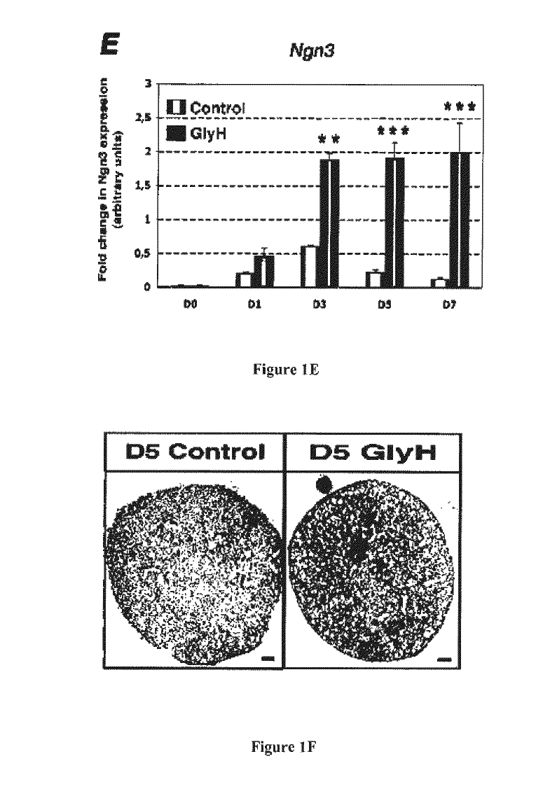

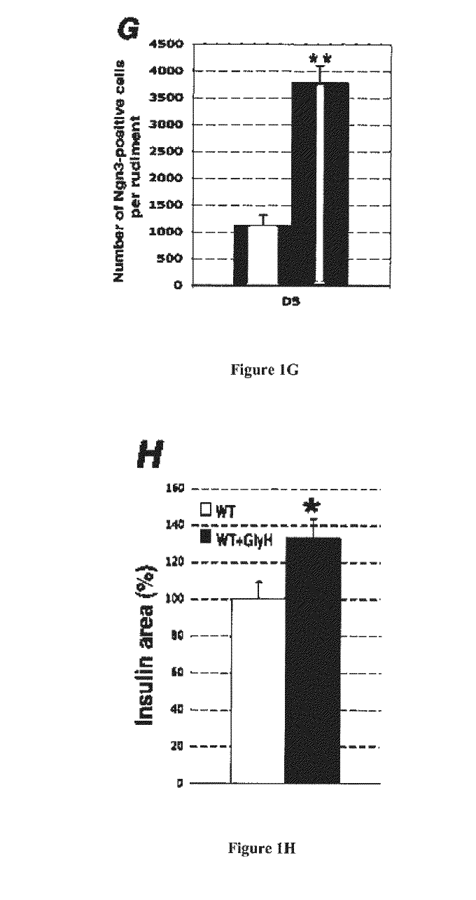

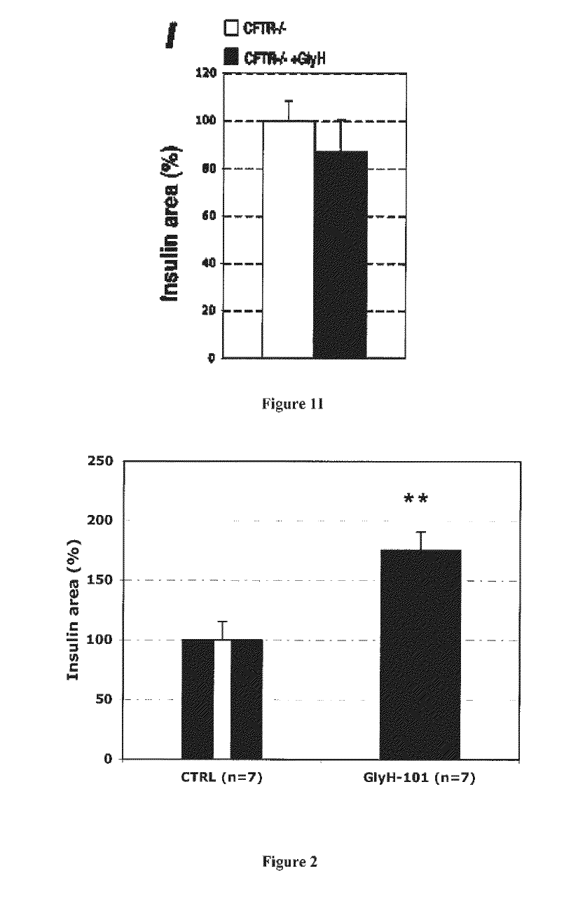

To determine the effects of GlyH-101 treatment on endocrine development, we focused on the expression pattern of insulin and the specific beta-cell marker, MafA. As shown in FIG. 1A-B, treating pancreases with GlyH-101 resulted in a major increase of insulin and MafA mRNA transcripts as early as D3 (more than twofold); the insulin expression being sixfold higher after 5 and 7 days of culture. The immunohistological analysis of insulin staining and the quantification of area occupied by insulin-positive cells further confirmed these previous results and showed a dramatic increase of insulin-expressing cells (FIG. 1C-D).

Given the observed increase of insulin-expressing cells with CFTR inhibitor treatment, we tested for an increased proliferation of such cells at D5 by adding BrdU to the medium during the last hour of culture. No differences were observed in the proliferative rate under the two conditions.

Thus, we asked wether the increase of the insulin⁺ cells was due to an enhancement of Ngn3 expression, the transcription factor which marks the proendocrine progenitors and is required for endocrine cell formation. By real-time PCR, we assessed mRNA levels and observed a strong increase in Ngn3 expression from D1, reaching a 8-fold increase at D5 under GlyH-101 treatment (FIG. 1-E). By immunochemistry (FIG. 1-F) and quantification of Ngn3⁺ cells at D5 (FIG. 1-G), we confirmed that GlyH-101 treatment amplified and maintained the pool of pancreatic endocrine progenitor expressing Ngn3, leading to an increase of the number of insulin-expressing cells. We did not observe any effects of GlyH-101 on exocrine differentiation. To further validate the ability of GlyH-101 to increase the number of pancreatic beta cells by targeting specifically CFTR channels, we first checked that the increase of insulin⁺ cell area observed in cultured Rat pancreas was found again in embryonic pancreases of WT mice (Cftr+/+) cultured with GlyH-101 (FIG. 1-H). In a second step, we compared the area occupied by insulin positive-cells in embryonic pancreases of Cftr knockout mice (Cftr−/−) cultured in presence or absence of GlyH-101. As shown in FIG. 1-I, and in contrast to the observed effects on WT pancreases, GlyH-101 was unable to modify insulin⁺ cell area in absence of CFTR channel. These results suggest strongly that the increase of beta cell differentiation induced by GlyH-101 is mediated by Cftr channel.

All together, our results indicate that GlyH-101, a specific inhibitor of CFTR channel mimics glibenclamide effects on Ngn3 cells and activates beta cell development by increasing the pool of endocrine progenitors NGN3+.

Discussion:

The sulfonylurea glibenclamide is an oral hypoglycemic agent used for the treatment of type 2 diabetes. It is known to stimulate insulin release by binding to the beta cell high affinity sulfonylurea receptor1 (SUR1), a subunit of the KATP channel. Although commonly used for the treatment of type 2 diabetes, glibenclamide was also successfully administrated to diabetic patients with KCNJ11 (gene encoding for the Kir.6.2 subunit of the KATP channel) mutations. In particular to young patients with permanent neonatal diabetes mellitus which were able therefore to stop insulin injections and to switch to sulfonylureas (Pearson et al. 2006; Codner et al. 2007; Stoy et al. 2008). We report here that glibenclamide, at a micromolar range and for a short period of 5 or 7 days, was able to expand dramatically the pool of the endocrine progenitor NGN3+ cells that further differentiate into insulin-expressing thus leading to a final increase in the number of beta cells without inducing any deleterious effects in the developing pancreas.

Our study revealed that 100 μM glibenclamide, even if added daily to the culture medium, is not deleterious for the embryonic pancreas. Indeed, glibenclamide did not affect the proliferation rate of the early PDX-1⁺ progenitors that remained high after one day of culture (31.23%±2.77 and 32.60%±4.19 PDX1⁺/BrdU⁺ cells respectively in the control and in the treated pancreases). Previous study have showed that any marked decrease in the proliferation of these initial PDX-1⁺ cells leads to the failure of the pancreatic growth and thus to an hypoplasia (Bhushan et al. 2001). Furthermore, the overall size of the pancreatic tissue after 7 days of glibenclamide treatment was not affected, as shown by the Hoechst staining of the nuclei. Although few informations are available on the in vitro effects of a such concentrations of glibenclamide, no toxicity nor apoptosis were observed in human liver cell line HepG2 cultured with 100 μM glibenclamide (Malhi et al. 2000). Finally, the fact that the exocrine and endocrine development were not altered by glibenclamide treatment suggests strongly that this sulfonylurea, even if used at these concentrations, is not deleterious for the pancreatic growth.

Although beta-cell development was similar in the 7 days-cultured pancreases in absence or in presence of glibenclamide (as shown by Znt8 and MafA expression and the surface area of PCSK1/PDX-1 positive cells), the glibenclamide-treated pancreases exhibited a low insulin content and a reduction of insulin gene transcription which was completely reverted only 2 days after glibenclamide removal. Recently, (Ling et al. 2006) have shown, that even if administrated at very low concentrations to an adult Rat during 2 days, glibenclamide was able to decrease the insulin mRNA; the same effect was observed again with 4 μM glibenclamide in isolated beta-cells. Nevertheless, they established that a subpopulation of these glibenclamide-treated beta-cells—have a higher rate of basal insulin synthesis.

The first major finding of the present study is the ability of glibenclamide to increase dramatically and to maintain Ngn3 gene expression (respectively seven- and threefold after 5 and 7 days of culture) leading to the amplification of the pool of pancreatic endocrine progenitors. Ngn3 plays a key role in islet differentiation and the importance of NGN3+ cells in generating the four endocrine cell types was highlighted with lineage tracing experiments (Gu et al. 2002)—as well as with NGN3-deficient mice (Gradwohl et al. 2000). It is known that pancreatic endocrine and exocrine precursor cells derive from the PDX1⁺ undifferentiated progenitors; and the increase of PDX1⁺ cells proliferation generates an amplification of Ngn3+ cells (Attali et al. 2007). In our study, it is unlikely that the proliferative rate of PDX1⁺ cells, similar in absence or in presence of glibenclamide, can account for the amplification of the pool of NGN3 cells observed in glibenclamide-treated pancreases. We can not exclude the hypothesis that glibenclamide enhanced the proliferation of these endocrine progenitors but NGN3⁺ cells have been reported to be a poor-proliferative cells (Attali et al. 2007; Haumaitre et al. 2008). Although several studies have advanced our knowledge (Apelqvist et al. 1999; Jensen et al. 2000), the tight regulation of Ngn3 expression remind still unclear.

The second major finding of this study is the dramatic increase of beta cell differentiation in the glibenclamide-treated pancreases as shown by the insulin staining after 9, 11 and 14 days of culture. It is unlikely that these beta cells originate by transdifferentiation of acinar or ductal tissues as no modifications were observed during the culture (FIG. 2 and data not shown). We can not rule out the hypothesis that glibenclamide promotes the number of replicating beta cells but it was shown that it had no significant effects on the proliferation of islet beta cells in vitro (Kwon et al. 2006) or in vivo (Guiot et al. 1994).

It is well established that pancreatic endocrine cell subtypes originate from NGN3⁺ endocrine precursors (Gradwohl et al. 2000). The fact that glucagon⁺ and insulin⁺ cell number (respectively FIGS. 3 and 5) was dramatically increased by glibenclamide treatment is consistent with the raise of NGN3⁺ cells number and the further differentiation of these endocrine progenitors into beta and alpha cells. Although NGN3 expression is crucial in the pancreatic regulatory network, it is not sufficient to allow the endocrine differentiation. It is known that the transcription factor NeuroD1/Beta2 is a direct target of Ngn3 (Huang et al. 2000). Moreover, it was shown that ectopic expression of NeuroD was able to induce endocrine differentiation (Gasa et al. 2004) while the lack of NeuroD resulted in a complete loss of endocrine cell development (Naya et al. 1997; Guillemain et al. 2007). Interestingly, we found that NeuroD1 was expressed twofold more in the glibenclamide-treated pancreases at D5 of culture (ie at the peak of Ngn3). Moreover, while Ngn3 expression began to decrease, NeuroD1 remained highly expressed suggesting strongly its involvement in the doubling of beta cells in the glibenclamide-treated pancreases. Finally, the fact that the removal of glibenclamide, just after the peak of Ngn3 (ie at D5), did not prevent the increase in the number of insulin positive cells suggest strongly that glibenclamide acts upstream of Ngn3. Thus, glibenclamide promotes the beta and alpha cell differentiation by increasing and maintaining Ngn3 expression without regulating the last steps of endocrine cell differentiation.

Because (i) the genes encoding the K_{ATP} channel SUR1 subunit and CFTR channel (respectively ABCC8 and ABCC7) belong to the same superfamily of ATP Binding Cassettes and (ii) glibenclamide inhibits CFTR channel (Sheppard and Robinson 1997; Yamazaki and Hume 1997; Gupta and Linsdell 2002), we investigated the glibenclamide CFTR-mediated effects on β cell differentiation by using GlyH-101, a specific inhibitor of CFTR channel. In the adult pancreas, CFTR works as a bicarbonate channel allowing the secretion of HCO3-rich fluid (Ishiguro et al. 2009). In the embryonic pancreas, CFTR mRNA is expressed throughout development (our results and (Hyde et al. 1997)) and was shown to label differentiated ductal cells (Hyde et al. 1997)—In this study, we show evidence that GlyH-101 targets specifically CFTR channel. Thus, the dramatic increase of Ngn3 expression and the subsequent beta cell differentiation observed in GlyH-101 treated-pancreases suggest strongly that (i) GlyH-101 mimics glibenclamide and (ii) the amplification of Ngn3+ endocrine progenitors could be related to an effect of glibenclamide on CFTR-expressing ductal cells. Interestingly, a subset of a ductal cells expressing the transcription factors Hnf1β or Hnf6 (Jacquemin et al. 2000; Maestro et al. 2003; Pierreux et al. 2006; Zhang et al. 2009) were shown to give rise to endocrine progenitors. In addition, more Ngn3 expressing cells at E15.5 were detected in transgenic mice overexpressing Hnf6 (Wilding Crawford et al. 2008). The plasticity of such embryonic pancreatic duct cells which gave rise to differentiated endocrine and ductal cells was highlighted by direct genetic labelling (Solar et al. 2009). Finally, it was reported that modulation of CFTR expression in lung progenitors affects their proliferation and their further differentiation (Larson et al. 2000).

In conclusion, we propose that inhibiting CFTR function or expression would represent a new approach to activate the pancreatic endocrine pathway.

### Example 2

Our previous results showed that GlyH was able to expand insulin-positive cell number in vitro.

To evaluate its effects in vivo, GlyH 101 was administrated to a E12.5 pregnant mice for 5 days at 10 mg/kg/day (according to Yang et al. 2008) in a saline DMSO solution (500 ml/injection) by intraperitoneal injection two times a day. The embryos were harvested at E18.5 and the pancreas fixed for histological immunostaining. The surface of insulin staining was quantified and normalized to the whole pancreatic tissue. As shown on FIG. 2, in vivo, GlyH 101 treatment increased beta cell mass.

### REFERENCES

Throughout this application, various references describe the state of the art to which this invention pertains. The disclosures of these references are hereby incorporated by reference into the present disclosure.
Apelqvist, A., H. Li, L. Sommer, P. Beatus, D. J. Anderson, T. Honjo, M. Hrabe de Angelis, U. Lendahl and H. Edlund (1999). “Notch signalling controls pancreatic cell differentiation.” Nature 400(6747): 877-81.
Attali, M., V. Stetsyuk, A. Basmaciogullari, V. Aiello, M. A. Zanta-Boussif, B. Duvillie and R. Scharfmann (2007). “Control of beta-cell differentiation by the pancreatic mesenchyme.” Diabetes 56(5): 1248-58.
Begum-Hasan, J., C. Polychronakos and H. Brill (2008). “Familial permanent neonatal diabetes with KCNJ11 mutation and the response to glyburide therapy—a three-year follow-up.” J Pediatr Endocrinol Metab 21(9): 895-903.
Bhushan, A., N. Itoh, S. Kato, J. P. Thiery, P. Czernichow, S. Bellusci and R. Scharfmann (2001). “Fgf10 is essential for maintaining the proliferative capacity of epithelial progenitor cells during early pancreatic organogenesis.” Development 128(24): 5109-17.
Chimienti, F., S. Devergnas, A. Favier and M. Seve (2004). “Identification and cloning of a beta-cell-specific zinc transporter, ZnT-8, localized into insulin secretory granules.” Diabetes 53(9): 2330-7.
Codner, E., S. E. Flanagan, F. Ugarte, H. Garcia, T. Vidal, S. Ellard and A. T. Hattersley (2007). “Sulfonylurea treatment in young children with neonatal diabetes: dealing with hyperglycemia, hypoglycemia, and sick days.” Diabetes Care 30(5): e28-9.
Duvillie, B., M. Attali, A. Bounacer, P. Ravassard, A. Basmaciogullari and R. Scharfmann (2006). “The mesenchyme controls the timing of pancreatic beta-cell differentiation.” Diabetes 55(3): 582-9.
Fischer-Lougheed, J., J. H. Liu, E. Espinos, D. Mordasini, C. R. Bader, D. Belin and L. Bernheim (2001). “Human myoblast fusion requires expression of functional inward rectifier Kir2.1 channels.” J Cell Biol 153(4): 677-86.
Flechtner, I., P. de Lonlay and M. Polak (2006). “Diabetes and hypoglycaemia in young children and mutations in the Kir6.2 subunit of the potassium channel: therapeutic consequences.” Diabetes Metab 32(6): 569-80.
Gasa, R., C. Mrejen, N. Leachman, M. Otten, M. Barnes, J. Wang, S. Chakrabarti, R. Mirmira and M. German (2004). “Proendocrine genes coordinate the pancreatic islet differentiation program in vitro.” Proc Natl Acad Sci USA 101(36): 13245-50.
Gloyn, A. L., E. R. Pearson, J. F. Antcliff, P. Proks, G. J. Bruining, A. S. Slingerland, N. Howard, S. Srinivasan, J. M. Silva, J. Molnes, E. L. Edghill, T. M. Frayling, I. K. Temple, D. Mackay, J. P. Shield, Z. Sumnik, A. van Rhijn, J. K. Wales, P. Clark, S. Gorman, J. Aisenberg, S. Ellard, P. R. Njolstad, F. M. Ashcroft and A. T. Hattersley (2004). “Activating mutations in the gene encoding the ATP-sensitive potassium-channel subunit Kir6.2 and permanent neonatal diabetes.” N Engl J Med 350(18): 1838-49.
Gradwohl, G., A. Dierich, M. LeMeur and F. Guillemot (2000). “neurogenin3 is required for the development of the four endocrine cell lineages of the pancreas.” Proc Natl Acad Sci USA 97(4): 1607-11.
Gu, G., J. Dubauskaite and D. A. Melton (2002). “Direct evidence for the pancreatic lineage: NGN3+ cells are islet progenitors and are distinct from duct progenitors.” Development 129(10): 2447-57.
Guillemain, G., G. Filhoulaud, G. Da Silva-Xavier, G. A. Rutter and R. Scharfmann (2007). “Glucose is necessary for embryonic pancreatic endocrine cell differentiation.” J Biol Chem 282(20): 15228-37.
Guiot, Y., J. C. Henquin and J. Rahier (1994). “Effects of glibenclamide on pancreatic beta-cell proliferation in vivo.” Eur J Pharmacol 261(1-2): 157-61.
Gupta, J. and P. Linsdell (2002). “Point mutations in the pore region directly or indirectly affect glibenclamide block of the CFTR chloride channel.” Pflugers Arch 443(5-6): 739-47.
Haumaitre, C., O. Lenoir and R. Scharfmann (2008). “Histone deacetylase inhibitors modify pancreatic cell fate determination and amplify endocrine progenitors.” Mol Cell Biol 28(20): 6373-83.
Heller, R. S., D. A. Stoffers, A. Liu, A. Schedl, E. B. Crenshaw, 3rd, O. D. Madsen and P. Serup (2004). “The role of Brn4/Pou3f4 and Pax6 in forming the pancreatic glucagon cell identity.” Dev Biol 268(1): 123-34.
Holmin, S., C. von Gertten, A. C. Sandberg-Nordqvist, U. Lendahl and T. Mathiesen (2001). “Induction of astrocytic nestin expression by depolarization in rats.” Neurosci Lett 314(3): 151-5.
Huang, H. P., M. Liu, H. M. El-Hodiri, K. Chu, M. Jamrich and M. J. Tsai (2000). “Regulation of the pancreatic islet-specific gene BETA2 (neuroD) by neurogenin 3.” Mol Cell Biol 20(9): 3292-307.
Hyde, K., C. J. Reid, S. J. Tebbutt, L. Weide, M. A. Hollingsworth and A. Harris (1997). “The cystic fibrosis transmembrane conductance regulator as a marker of human pancreatic duct development.” Gastroenterology 113(3): 914-9.
Ishiguro, H., M. C. Steward, S, Naruse, S. B. Ko, H. Goto, R. M. Case, T. Kondo and A. Yamamoto (2009). “CFTR functions as a bicarbonate channel in pancreatic duct cells.” J Gen Physiol 133(3): 315-26.
Jacquemin, P., S. M. Durviaux, J. Jensen, C. Godfraind, G. Gradwohl, F. Guillemot, O. D. Madsen, P. Carmeliet, M. Dewerchin, D. Collen, G. G. Rousseau and F. P. Lemaigre (2000). “Transcription factor hepatocyte nuclear factor 6 regulates pancreatic endocrine cell differentiation and controls expression of the proendocrine gene ngn3.” Mol Cell Biol 20(12): 4445-54.
Jensen, J., R. S. Heller, T. Funder-Nielsen, E. E. Pedersen, C. Lindsell, G. Weinmaster, O. D. Madsen and P. Serup (2000). “Independent development of pancreatic alpha- and beta-cells from neurogenin3-expressing precursors: a role for the notch pathway in repression of premature differentiation.” Diabetes 49(2): 163-76.
Kelly, M., S. Trudel, F. Brouillard, F. Bouillaud, J. Colas, T. Nguyen-Khoa, M. Ollero, A. Edelman and J. Fritsch (2010). “Cystic fibrosis transmembrane regulator inhibitors CFTR(inh)-172 and GlyH-101 target mitochondrial functions, independently of chloride channel inhibition.” J Pharmacol Exp Ther 333(1): 60-9.
Kilic, G., J. Wang and B. Sosa-Pineda (2006). “Osteopontin is a novel marker of pancreatic ductal tissues and of undifferentiated pancreatic precursors in mice.” Dev Dyn 235(6): 1659-67.
Kwon, G., C. A. Marshall, H. Liu, K. L. Pappan, M. S. Remedi and M. L. McDaniel (2006). “Glucose-stimulated DNA synthesis through mammalian target of rapamycin (mTOR) is regulated by KATP channels: effects on cell cycle progression in rodent islets.” J Biol Chem 281(6): 3261-7.
Larson, J. E., J. B. Delcarpio, M. M. Farberman, S. L. Morrow and J. C. Cohen (2000). “CFTR modulates lung secretory cell proliferation and differentiation.” Am J Physiol Lung Cell Mol Physiol 279(2): L333-41.
Ling, Z., Q. Wang, G. Stange, P. In't Veld and D. Pipeleers (2006). “Glibenclamide treatment recruits beta-cell subpopulation into elevated and sustained basal insulin synthetic activity.” Diabetes 55(1): 78-85.
Livak, K. J. and T. D. Schmittgen (2001). “Analysis of relative gene expression data using real-time quantitative PCR and the 2(-Delta Delta C(T)) Method.” Methods 25(4): 402-8.
Maestro, M. A., S. F. Boj, R. F. Luco, C. E. Pierreux, J. Cabedo, J. M. Servitja, M. S. German, G. G. Rousseau, F. P. Lemaigre and J. Ferrer (2003). “Hnf6 and Tcf2 (MODY5) are linked in a gene network operating in a precursor cell domain of the embryonic pancreas.” Hum Mol Genet. 12(24): 3307-14.
Malhi, H., A. N. Irani, P. Rajvanshi, S. O. Suadicani, D. C. Spray, T. V. McDonald and S. Gupta (2000). “KATP channels regulate mitogenically induced proliferation in primary rat hepatocytes and human liver cell lines. Implications for liver growth control and potential therapeutic targeting.” J Biol Chem 275(34): 26050-7.
Mellor, J. R., D. Merlo, A. Jones, W. Wisden and A. D. Randall (1998). “Mouse cerebellar granule cell differentiation: electrical activity regulates the GABAA receptor alpha 6 subunit gene.” J Neurosci 18(8): 2822-33.
Miki, T., T. Iwanaga, K. Nagashima, Y. Ihara and S. Seino (2001). “Roles of ATP-sensitive K+ channels in cell survival and differentiation in the endocrine pancreas.” Diabetes 50 Suppl 1: S48-51.
Miralles, F., P. Czernichow and R. Scharfmann (1998). “Follistatin regulates the relative proportions of endocrine versus exocrine tissue during pancreatic development.” Development 125(6): 1017-24.
Muanprasat, C., N. D. Sonawane, D. Salinas, A. Taddei, L. J. Galietta and A. S. Verkman (2004). “Discovery of glycine hydrazide pore-occluding CFTR inhibitors: mechanism, structure-activity analysis, and in vivo efficacy.” J Gen Physiol 124(2): 125-37.
Naya, F. J., H. P. Huang, Y. Qiu, H. Mutoh, F. J. DeMayo, A. B. Leiter and M. J. Tsai (1997). “Diabetes, defective pancreatic morphogenesis, and abnormal enteroendocrine differentiation in BETA2/neuroD-deficient mice.” Genes Dev 11(18): 2323-34.
Njolstad, P. R., O. Sovik, A. Cuesta-Munoz, L. Bjorkhaug, O. Massa, F. Barbetti, D. E. Undlien, C. Shiota, M. A. Magnuson, A. Molven, F. M. Matschinsky and G. I. Bell (2001). “Neonatal diabetes mellitus due to complete glucokinase deficiency.” N Engl J Med 344(21): 1588-92.
Ohlsson, H., K. Karlsson and T. Edlund (1993). “IPF1, a homeodomain-containing transactivator of the insulin gene.” Embo J 12(11): 4251-9.
Pearson, E. R., I. Flechtner, P. R. Njolstad, M. T. Malecki, S. E. Flanagan, B. Larkin, F. M. Ashcroft, I. Klimes, E. Codner, V. Iotova, A. S. Slingerland, J. Shield, J. J. Robert, J. J. Hoist, P. M. Clark, S. Ellard, O. Sovik, M. Polak and A. T. Hattersley (2006). “Switching from insulin to oral sulfonylureas in patients with diabetes due to Kir6.2 mutations.” N Engl J Med 355(5): 467-77.
Pierreux, C. E., A. V. Poll, C. R. Kemp, F. Clotman, M. A. Maestro, S. Cordi, J. Ferrer, L. Leyns, G. G. Rousseau and F. P. Lemaigre (2006). “The transcription factor hepatocyte nuclear factor-6 controls the development of pancreatic ducts in the mouse.” Gastroenterology 130(2): 532-41.
Polak, M., A. Dechaume, H. Cave, R. Nimri, H. Crosnier, V. Sulmont, M. de Kerdanet, R. Scharfmann, Y. Lebenthal, P. Froguel and M. Vaxillaire (2008). “Heterozygous missense mutations in the insulin gene are linked to permanent diabetes appearing in the neonatal period or in early infancy: a report from the French ND (Neonatal Diabetes) Study Group.” Diabetes 57(4): 1115-9.
Schultz, B. D., A. D. DeRoos, C. J. Venglarik, A. K. Singh, R. A. Frizzell and R. J. Bridges (1996). “Glibenclamide blockade of CFTR chloride channels.” Am J Physiol 271(2 Pt 1): L192-200.
Seghers, V., M. Nakazaki, F. DeMayo, L. Aguilar-Bryan and J. Bryan (2000). “Sur1 knockout mice. A model for K(ATP) channel-independent regulation of insulin secretion.” J Biol Chem 275(13): 9270-7.
Sheppard, D. N. and K. A. Robinson (1997). “Mechanism of glibenclamide inhibition of cystic fibrosis transmembrane conductance regulator Cl-channels expressed in a murine cell line.” J Physiol 503 (Pt 2): 333-46.
Sheppard, D. N. and M. J. Welsh (1992). “Effect of ATP-sensitive K+ channel regulators on cystic fibrosis transmembrane conductance regulator chloride currents.” J Gen Physiol 100(4): 573-91.
Shimomura, K., F. Horster, H. de Wet, S. E. Flanagan, S. Ellard, A. T. Hattersley, N. I. Wolf, F. Ashcroft and F. Ebinger (2007). “A novel mutation causing DEND syndrome: a treatable channelopathy of pancreas and brain.” Neurology 69(13): 1342-9.
Shiota, C., O. Larsson, K. D. Shelton, M. Shiota, A. M. Efanov, M. Hoy, J. Lindner, S. Kooptiwut, L. Juntti-Berggren, J. Gromada, P. O. Berggren and M. A. Magnuson (2002). “Sulfonylurea receptor type 1 knock-out mice have intact feeding-stimulated insulin secretion despite marked impairment in their response to glucose.” J Biol Chem 277(40): 37176-83.
Slingerland, A. S, and A. T. Hattersley (2006a). “Activating mutations in the gene encoding Kir6.2 alter fetal and postnatal growth and also cause neonatal diabetes.” J Clin Endocrinol Metab 91(7): 2782-8.
Slingerland, A. S., R. Nuboer, M. Hadders-Algra, A. T. Hattersley and G. J. Bruining (2006b). “Improved motor development and good long-term glycaemic control with sulfonylurea treatment in a patient with the syndrome of intermediate developmental delay, early-onset generalised epilepsy and neonatal diabetes associated with the V59M mutation in the KCNJ11 gene.” Diabetologia 49(11): 2559-63.
Slingerland, A. S., W. Hurkx, K. Noordam, S. E. Flanagan, J. W. Jukema, L. C. Meiners, G. J. Bruining, A. T. Hattersley and M. Hadders-Algra (2008). “Sulphonylurea therapy improves cognition in a patient with the V59M KCNJ11 mutation.” Diabet Med 25(3): 277-81.
Snouwaert, J. N., K. K. Brigman, A. M. Latour, N. N. Malouf, R. C. Boucher, O. Smithies and B. H. Koller (1992). “An animal model for cystic fibrosis made by gene targeting.” Science 257(5073): 1083-8.
Solar, M., C. Cardalda, I. Houbracken, M. Martin, M. A. Maestro, N. De Medts, X. Xu, V. Grau, H. Heimberg, L. Bouwens and J. Ferrer (2009). “Pancreatic exocrine duct cells give rise to insulin-producing beta cells during embryogenesis but not after birth.” Dev Cell 17(6): 849-60.
Stoffers, D. A., N. T. Zinkin, V. Stanojevic, W. L. Clarke and J. F. Habener (1997). “Pancreatic agenesis attributable to a single nucleotide deletion in the human IPF1 gene coding sequence.” Nat Genet. 15(1): 106-10.
Stoy, J., S. A. Greeley, V. P. Paz, H. Ye, A. N. Pastore, K. B. Skowron, R. B. Lipton, F. R. Cogen, G. I. Bell and L. H. Philipson (2008). “Diagnosis and treatment of neonatal diabetes: a United States experience.” Pediatr Diabetes 9(5): 450-9.
Vaxillaire, M., C. Populaire, K. Busiah, H. Cave, A. L. Gloyn, A. T. Hattersley, P. Czernichow, P. Froguel and M. Polak (2004). “Kir6.2 mutations are a common cause of permanent neonatal diabetes in a large cohort of French patients.” Diabetes 53(10): 2719-22.
Wilding Crawford, L., E. Tweedie Ables, Y. A. Oh, B. Boone, S. Levy and M. Gannon (2008). “Gene expression profiling of a mouse model of pancreatic islet dysmorphogenesis.” PLoS One 3(2): e1611.
Winarto, A., T. Miki, S. Seino and T. Iwanaga (2001). “Morphological changes in pancreatic islets of KATP channel-deficient mice: the involvement of KATP channels in the survival of insulin cells and the maintenance of islet architecture.” Arch Histol Cytol 64(1): 59-67.
Yamazaki, J. and J. R. Hume (1997). “Inhibitory effects of glibenclamide on cystic fibrosis transmembrane regulator, swelling-activated, and Ca(2+)-activated Cl-channels in mammalian cardiac myocytes.” Circ Res 81(1): 101-9.
Zhang, C., T. Moriguchi, M. Kajihara, R. Esaki, A. Harada, H. Shimohata, H. Oishi, M. Hamada, N. Morito, K. Hasegawa, T. Kudo, J. D. Engel, M. Yamamoto and S. Takahashi (2005). “MafA is a key regulator of glucose-stimulated insulin secretion.” Mol Cell Biol 25(12): 4969-76.
Zhang, H., E. T. Ables, C. F. Pope, M. K. Washington, S. Hipkens, A. L. Means, G. Path, J. Seufert, R. H. Costa, A. B. Leiter, M. A. Magnuson and M. Gannon (2009).

“Multiple, temporal-specific roles for HNF6 in pancreatic endocrine and ductal differentiation.” Mech Dev.

1-5. (canceled)

6. A method for screening a drug for the treatment of disorders of glucose homeostasis, said method comprising contacting a test compound with a cystic fibrosis transmembrane regulator (CFTR) protein or gene and determining the ability of said test compound to inhibit the expression and/or activity of said gene or protein.

7. An in vitro method for increasing the pool of Ngn3+ endocrine progenitor cells obtained from stem cells, wherein said method comprises the step of contacting stem cells having the capacity to differentiate into pancreatic endocrine cells with a CFTR inhibitor or an inhibitor of CFTR gene expression.

8. An in vitro method for increasing the number of pancreatic endocrine cells obtained from stem cells, wherein said method comprises the step of contacting stem cells having the capacity to differentiate into pancreatic endocrine cells with a CFTR inhibitor or an inhibitor of CFTR gene expression.

9. An in vitro method for increasing the β cell mass obtained from stem cells, wherein said method comprises the step of contacting stem cells having the capacity to differentiate into pancreatic endocrine cells with a CFTR inhibitor or an inhibitor of CFTR gene expression.

10. An in vitro method for obtaining pancreatic endocrine cells, wherein said method comprises the step of contacting stem cells having the capacity to differentiate into pancreatic endocrine cells with a CFTR inhibitor or an inhibitor of CFTR gene expression.

11. A method of testing a subject thought to have or be predisposed to having disorders of glucose homeostasis, which comprises the step of analyzing a sample of interest from said subject by:
(i) detecting a mutation in a CFTR gene and/or its associated promoter, and/or
(ii) analyzing expression of the CFTR gene.

12. A method of treating disorders of glucose homeostasis in a patient in need thereof, comprising
administering to said patient a CTFR inhibitor in a therapeutically effective amount sufficient to treat said disorder of glucose homeostasis.

13. The method of claim 12, wherein said CFTR inhibitor is selected from the group consisting of aptamers, antibodies and small organic molecules.

14. The method of claim 13, wherein said CFTR inhibitor is N-2-napthalenyl-[(3,5-dibromo-2,4-dihydroxyphenyl)methylene]glycine hvdrazide

15. A method of treating disorders of glucose homeostasis in a patient in need thereof, comprising
administering to said patient an inhibitor of CFTR gene expression in a therapeutically effective amount sufficient to treat said disorder of glucose homeostasis.

16. The method of claim 15, wherein said wherein said inhibitor of CFTR gene expression is selected from the group consisting of antisense RNA or DNA molecules, small inhibitory RNAs (siRNAs), short hairpin RNA and ribozymes.

17. The method of claim 12, wherein said step of administering is carried out by providing said patient with a biodegradable implant comprising said CTFR inhibitor
